(19) United States
(12) Patent Application Publication
(10) Pub. No.: US 2015/0133460 A1
(43) Pub. Date: May 14, 2015

(54) LACTATE SALT OF 4-[6-METHOXY-7-(3-PIPERIDIN-1-YL-PROPOXY)QUINAZOLIN-4-YL]PIPERAZINE-1-CARBOXYLIC ACID(4-ISOPROPOXYPHENYL)-AMIDE AND PHARMACEUTICAL COMPOSITIONS THEREOF FOR THE TREATMENT OF CANCER AND OTHER DISEASES OR DISORDERS

(71) Applicant: Millennium Phamaceuticals, Inc., Cambridge, MA (US)

(72) Inventors: Ian Armitage, Stoneham, MA (US); Abu J. Ferdous, Winchester, MA (US); Michael E. Bourland, Belmont, MA (US); Craig S.J. Boyle, Swavesey (GB); Martin Ian Cooper, Foxton (GB); Marianne Langston, North Andover, MA (US)

(73) Assignee: Millennium Phamaceuticals, Inc., Cambridge, MA (US)

(21) Appl. No.: 14/076,958

(22) Filed: Nov. 11, 2013

(51) Int. Cl. C07D 239/94; A61K 9/28; A61K 9/20

(52) U.S. Cl. CPC C07D 239/94; A61K 9/2095; A61K 9/2893
USPC 514/252.17; 544/293; 264/118

(57) ABSTRACT

This invention provides a compound of formula (I):

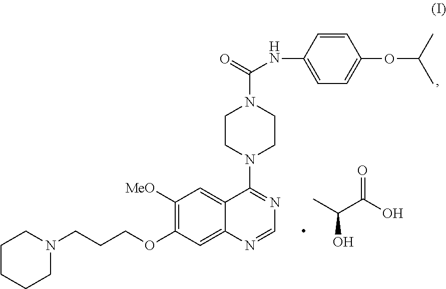

or a crystalline form thereof, or a pharmaceutical composition thereof, or an oral pharmaceutical dosage form thereof; processes for the synthesis or manufacture of the compound of formula (I), or a crystalline form thereof, or a pharmaceutical composition thereof, or an oral pharmaceutical dosage form thereof; and the use of said compound, or a crystalline form thereof, or a pharmaceutical composition thereof, or an oral pharmaceutical dosage form thereof, for the treatment of patients suffering from or subject to diseases, disorders or conditions involving cell survival, proliferation, and migration, including cardiovascular disease (e.g., arteriosclerosis and vascular reobstruction), cancer, (e.g., AML and malignant glioma) glomerulosclerosis, fibrotic disease and inflammation.

The present application is a continuation of U.S. application Ser. No. 12/622,617, filed Nov. 20, 2009 (now Published), and claims priority from U.S. Provisional application Ser. No. 61/199,888, filed Nov. 21, 2008 (now expired), and U.S. Provisional application Ser. No. 61/210,398 filed Mar. 17, 2009 (now expired); both herein incorporated by reference in their entirety.

### FIELD OF THE INVENTION

The present invention relates to the (L)-lactate salt of 4-[6-methoxy-7-(3-piperidin-1-yl-propoxy)quinazolin-4-yl]piperazine-1-carboxylic acid(4-isopropoxyphenyl)-amide of formula (I):

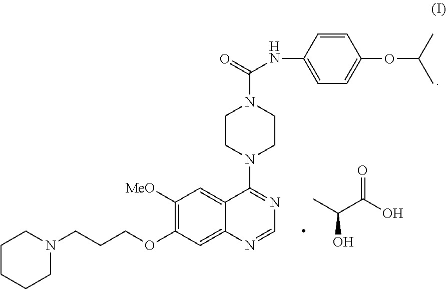

or a crystalline form thereof.

The present invention is also directed to a process for the synthesis of the compound of formula (I), or a crystalline form thereof. The present invention is also directed to pharmaceutical compositions of 4-[6-methoxy-7-(3-piperidin-1-yl-propoxy)quinazolin-4-yl]piperazine-1-carboxylic acid(4-isopropoxyphenyl)-amide lactate salt of formula (I), or a crystalline form thereof. The invention is also directed to methods of use of the compound of formula (I), or a crystalline form thereof, or pharmaceutical compositions thereof for the treatment of cancer and other disorders.

### BACKGROUND OF THE INVENTION

The compound 4-[6-methoxy-7-(3-piperidin-1-yl-propoxy)quinazolin-4-yl]piperazine-1-carboxylic acid(4-isopropoxyphenyl)-amide of formula (II) is a small molecule, ATP-competitive and reversible inhibitor of Type III receptor tyrosine kinases (RTKs). In vitro, the compound of formula (II) inhibits Flt-3, c-Kit and PDGFR-beta with a median IC50 of approximately 30 nM. In cellular assays, the compound of formula (II) inhibited the autophosphorylation of these receptors with an IC50 of approximately 200 nM (Pandey et al., J. Med. Chem. 2002, 45:3772-3793).

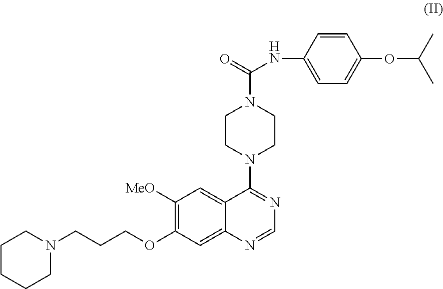 [SMILES: COc1cc2c(N3CCN(C(=O)Nc4ccc(OC(C)C)cc4)CC3)ncnc2cc1OCCCN1CCCCC1]

Approximately 30% of patients with acute myelogenous leukemia (AML) have a mutation in their Flt-3 gene, specifically an internal tandem duplication (ITD), which may be implicated in the growth and survival of the leukemia. In preclinical studies, the compound of formula (II) selectively killed human Flt-3/ITD-positive AML cancer cells (Kelly et al., Cancer Cell 2002, 1(5):421-432). The sulfate salt of the compound of formula (II) has been studied in clinical trials in AML patients (DeAngelo et al., Blood 2006, 108:3674-3681).

Malignant gliomas are the most common form of primary brain tumors in adults. Of the malignant gliomas, glioblastoma multiformes (GBM) account for approximately 60-70% of malignant gliomas, anaplastic astrocytomas account for 10-15%, anaplastic oligodendrogliomas, and anaplastic oligoastrocytomas account for 10%, and less commonly occurring tumors account for the rest (Kesari et al., Current Neurology and Neuroscience Reports 2005, 5:186-187).

Dysregulated autocrine PDGF stimulation is thought to contribute both to the early transformation events and to the maintenance of glioma tumorigenesis. The PDGFR-alpha subunit is overexpressed in virtually all glioma cell lines, and primary cultures of malignant gliomas, and the PDGFR-beta subunit is frequently expressed within glioma tumor cells and endothelial cells. (Kesari et al., Current Neurology and Neuroscience Reports 2005, 5:186-187). c-Kit is also expressed by a percentage of primary glioblastoma tumors (Gomes et al., Cell Oncol. 2007, 29 (5):399-408).

It has also been shown that PDGFR inhibition significantly enhances VEGFR anti-angiogenic activity (Shen et al., Biochem Biophys Res Commun 2007, 357(4):1142-1147). A combination of mouse monoclonal antibodies specific for PDGFR and VEGFR showed a tumor regression in 58% of mice compared to 18% for VEGFR antibody alone in two different xenograft models (pancreatic and non-small cell lung cancer).

WO 02/016351, U.S. Ser. No. 05/101,609, U.S. Ser. No. 05/288,297, and U.S. Pat. No. 6,982,266 disclose substituted quinazoline compounds that exhibit an inhibitory effect on type III tyrosine kinases, especially Flt-3, PDGFR, and c-Kit. These applications additionally disclose methods for the preparation of these compounds, pharmaceutical compositions containing these compounds, and methods for the prophylaxis and therapy of diseases, disorders, or conditions associated with an increased tyrosine kinase activity, including, but not limited to, cardiovascular disease (e.g., arteriosclerosis and vascular reobstruction), cancer (e.g., leukemia such as acute lymphocytic leukemia, or malignant glioma), glomerulosclerosis fibrotic diseases and inflammation, and general treatment of cell-proliferative diseases.

4-[6-methoxy-7-(3-piperidin-1-yl-propoxy)quinazolin-4-yl]piperazine-1-carboxylic acid(4-isopropoxyphenyl)-amide (II) is described in WO 02/016351, U.S. Ser. No. 05/101,609, U.S. Ser. No. 05/288,297, U.S. Pat. No. 6,982,266, and Pandey et al., J. Med. Chem. 2002, 45:3772-3793. WO 07/012402 describes crystalline forms of the sulfate salt of 4-[6-methoxy-7-(3-piperidin-1-yl-propoxy)quinazolin-4-yl]piperazine-1-carboxylic acid(4-isopropoxyphenyl)-amide.

In the Pandey et al. reported synthesis, 4-[6-methoxy-7-(3-piperidin-1-yl-propoxy)quinazolin-4-yl]piperazine-1-carboxylic acid(4-isopropoxyphenyl)-amide of formula (II) is isolated as the hydrochloride salt after being dried in vacuo. WO 02/36587 describes a process for the synthesis of the compound of formula (II), and a method for making the compound of formula (II) as a sulfate salt. WO 07/012402 describes crystalline forms of the sulfate salt of 4-[6-methoxy-7-(3-piperidin-1-yl-propoxy)quinazolin-4-yl]piperazine-1-carboxylic acid(4-isopropoxyphenyl)-amide.

These applications and publications do not disclose other salts or crystalline forms of 4-[6-methoxy-7-(3-piperidin-1-yl-propoxy)quinazolin-4-yl]piperazine-1-carboxylic acid(4-isopropoxyphenyl)-amide (II).

The large-scale manufacturing of a pharmaceutical composition poses many challenges to the chemist and chemical engineer. While many of these challenges relate to the handling of large quantities of reagents and control of large-scale reactions, the handling of the final product poses special challenges linked to the nature of the final active product itself. Not only must the product be prepared in high yield, be stable, and capable of ready isolation, the product must possess properties that are suitable for the types of pharmaceutical preparations in which they are likely to be ultimately used. The stability of the active ingredient of the pharmaceutical preparation must be considered during each step of the manufacturing process, including the synthesis, isolation, bulk storage, pharmaceutical formulation and long-term formulation. Each of these steps may be impacted by various environmental conditions of temperature and humidity.

The pharmaceutically active substance used to prepare the pharmaceutical compositions should be as pure as possible, and its stability on long-term storage must be guaranteed under various environmental conditions. These properties are absolutely essential to prevent the appearance of unintended degradation products in pharmaceutical compositions, which degradation products may be potentially toxic or result simply in reducing the potency of the composition.

A primary concern for the manufacture of large-scale pharmaceutical compounds is that the active substance should have a stable crystalline morphology to ensure consistent processing parameters and pharmaceutical quality. If an unstable crystalline form is used, crystal morphology may change during manufacture and/or storage resulting in quality control problems, and formulation irregularities. Such a change may affect the reproducibility of the manufacturing process and thus lead to final formulations which do not meet the high quality and stringent requirements imposed on formulations of pharmaceutical compositions. In this regard, it should be generally borne in mind that any change to the solid state of a pharmaceutical composition which can improve its physical and chemical stability gives a significant advantage over less stable forms of the same drug.

When a compound crystallizes from a solution or slurry, it may crystallize with different spatial lattice arrangements, a property referred to as “polymorphism.” Each of the crystal forms is a “polymorph.” While polymorphs of a given substance have the same chemical composition, they may differ from each other with respect to one or more physical properties, such as solubility and dissociation, true density, melting point, crystal shape, compaction behavior, flow properties, and/or solid state stability.

As described generally above, the polymorphic behavior of drugs can be of great importance in pharmacy and pharmacology. The differences in physical properties exhibited by polymorphs affect practical parameters such as storage stability, compressibility and density (important in formulation and product manufacturing), and dissolution rates (an important factor in determining bio-availability). Differences in stability can result from changes in chemical reactivity (e.g., differential oxidation, such that a dosage form discolors more rapidly when it is one polymorph than when it is another polymorph) or mechanical changes (e.g., tablets crumble on storage as a kinetically favored polymorph converts to thermodynamically more stable polymorph) or both (e.g., tablets of one polymorph are more susceptible to breakdown at high humidity). In addition, the physical properties of the crystal may be important in processing: for example, one polymorph might be more likely to form solvates that cause the solid form to aggregate and increase the difficulty of solid handling, or might be difficult to filter and wash free of impurities (i.e., particle shape and size distribution might be different between one polymorph relative to other).

While drug formulations having improved chemical and physical properties are desired, there is no predictable means for preparing new drug forms (e.g., polymorphs) of existing molecules for such formulations. These new forms would provide consistency in physical properties over a range of environments common to manufacturing and composition usage. More particularly, there is a need for an inhibitor of type III tyrosine kinases, that operates through the inhibition of such tyrosine kinases, especially Flt-3, PDGFR, and c-Kit. Such an inhibitor should have utility in treating a patient suffering from or subject to type III tyrosine kinases mediated pathological (diseases) conditions, including cardiovascular disease (e.g., arteriosclerosis and vascular reobstruction), cancer (e.g., leukemia such as acute myelogenous leukemia; or malignant glioma), glomerulosclerosis fibrotic diseases and inflammation, and general treatment of cell-proliferative diseases, as well as having properties suitable for large-scale manufacturing and formulation.

There is a need to develop additional salts of the compound of formula (II), especially those that are useful for large-scale manufacturing, pharmaceutical formulations, and storage. There is also a need to develop pharmaceutical formulations of the compound of formula (II), or a salt thereof, or a crystalline form thereof, especially those that allow for a high-drug loading, are convenient to administer and are stable.

### DESCRIPTION OF THE INVENTION

One aspect of the present invention provides the (L)-lactate salt of 4-[6-methoxy-7-(3-piperidin-1-yl-propoxy)quinazolin-4-yl]piperazine-1-carboxylic acid (4-isopropoxyphenyl)-amide of formula (I), or a crystalline form thereof the possible crystalline forms being described herein.

In another aspect, the present invention provides processes for the synthesis of the (L)-lactate salt of 4-[6-methoxy-7-(3-piperidin-1-yl-propoxy)quinazolin-4-yl]piperazine-1-carboxylic acid (4-isopropoxyphenyl)-amide (I), or a crystalline form thereof. Other embodiments of the invention are directed to said processes, wherein the Lactate Salt is a crystalline form, the possible crystalline forms being described herein.

In another aspect, the present invention provides pharmaceutical compositions comprising a pharmaceutically acceptable carrier or diluent, and the (L)-lactate salt of 4-{6-methoxy-7-(3-piperidin-1-yl-propoxy)quinazolin-4-yl]piperazine-1-carboxylic acid (4-isopropoxyphenyl)-amide (I), or a crystalline form thereof. Other embodiments of the invention are directed to said pharmaceutical compositions, wherein the Lactate Salt is a crystalline form; the possible crystalline forms being described herein.

In another aspect, the present invention is provides a pharmaceutical composition comprising the compound of formula (I), or a crystalline form thereof, suitable for the bulk production of an oral pharmaceutical dosage form.

In another aspect, the present invention provides a pharmaceutical composition comprising the compound of formula (I), or a crystalline form thereof, suitable for the bulk production of tablets.

In another aspect, the present invention provides a pharmaceutical composition, comprising the compound of formula (I), or a crystalline form thereof, a lubricant, a disintegrant, a filler, and a glidant.

In another aspect, the present invention provides an oral pharmaceutical dosage form with high drug loading, comprising the compound of formula (I), or a crystalline form thereof, as the active ingredient.

In another aspect, the present invention provides a process for the bulk production of the oral pharmaceutical dosage form of the compound of formula (I), or a crystalline form thereof.

In another aspect, the present invention provides methods for the use of the pharmaceutical composition of the compound of formula (I), or a crystalline form thereof, for the treatment of patients suffering from or subject to diseases, disorders or conditions involving cell survival, proliferation, and migration, including cardiovascular disease (e.g., arteriosclerosis and vascular reobstruction), cancer, (e.g., AML and malignant glioma) glomerulosclerosis, fibrotic disease and inflammation.

The present invention shall be more fully discussed with the aid of the following figures and detailed description below.

### BRIEF DESCRIPTION OF THE DRAWINGS

FIG. 1 is a powder X-ray diffractogram (XRPD) of 4-[6-methoxy-7-(3-piperidin-1-yl-propoxy)quinazolin-4-yl]piperazine-1-carboxylic acid(4-isopropoxyphenyl)-amide (L)-lactate Form 1.

FIG. 2 is a differential scanning calorimetry (DSC)/thermal gravimetric analysis (TGA) profile for 4-[6-methoxy-7-(3-piperidin-1-yl-propoxy)quinazolin-4-yl]piperazine-1-carboxylic acid(4-isopropoxyphenyl)-amide (L)-lactate Form 1.

FIG. 3 is a gravimetric vapor sorption (GVS) profile for 4-[6-methoxy-7-(3-piperidin-1-yl-propoxy)quinazolin-4-yl]piperazine-1-carboxylic acid(4-isopropoxyphenyl)-amide (L)-lactate Form 1.

FIG. 4 is a powder X-ray diffractogram (XRPD) of 4-[6-methoxy-7-(3-piperidin-1-yl-propoxy)quinazolin-4-yl]piperazine-1-carboxylic acid(4-isopropoxyphenyl)-amide (L)-lactate Form 2.

FIG. 5 is a differential scanning calorimetry (DSC) profile for 4-[6-methoxy-7-(3-piperidin-1-yl-propoxy)quinazolin-4-yl]piperazine-1-carboxylic acid(4-isopropoxyphenyl)-amide (L)-lactate Form 2

FIG. 6 is a gravimetric vapor sorption (GVS) profile of 4-[6-methoxy-7-(3-piperidin-1-yl-propoxy)quinazolin-4-yl]piperazine-1-carboxylic acid(4-isopropoxyphenyl)-amide (L)-lactate Form 2.

FIG. 7 is a powder X-ray diffractogram (XRPD) of 4-[6-methoxy-7-(3-piperidin-1-yl-propoxy)quinazolin-4-yl]piperazine-1-carboxylic acid(4-isopropoxyphenyl)-amide (L)-lactate Form 3.

FIG. 8 is a differential scanning calorimetry (DSC)/thermal gravimetric analysis (TGA) profile for 4-[6-methoxy-7-(3-piperidin-1-yl-propoxy)quinazolin-4-yl]piperazine-1-carboxylic acid(4-isopropoxyphenyl)-amide (L)-lactate Form 3.

FIG. 9 is a gravimetric vapor sorption (GVS) profile for 4-[6-methoxy-7-(3-piperidin-1-yl-propoxy)quinazolin-4-yl]piperazine-1-carboxylic acid(4-isopropoxyphenyl)-amide (L)-lactate Form 3.

FIG. 10 is a powder X-ray diffractogram (XRPD) of 4-[6-methoxy-7-(3-piperidin-1-yl-propoxy)quinazolin-4-yl]piperazine-1-carboxylic acid(4-isopropoxyphenyl)-amide (L)-lactate Form 4.

FIG. 11 is a differential scanning calorimetry (DSC)/thermal gravimetric analysis (TGA) profile for 4-[6-methoxy-7-(3-piperidin-1-yl-propoxy)quinazolin-4-yl]piperazine-1-carboxylic acid(4-isopropoxyphenyl)-amide (L)-lactate Form 4.

FIG. 12 is a gravimetric vapor sorption (GVS) for 4-[6-methoxy-7-(3-piperidin-1-yl-propoxy)quinazolin-4-yl]piperazine-1-carboxylic acid(4-isopropoxyphenyl)-amide (L)-lactate Form 4.

### DETAILED DESCRIPTION OF THE INVENTION

### Definitions and Abbreviations

As used above, and throughout the description of the invention, the following terms and phrases, unless otherwise indicated, shall be understood to have the following meanings.

As used herein, the phrase “Lactate Salt” is meant to describe the (L)-lactate salt of 4-[6-methoxy-7-(3-piperidin-1-yl-propoxy)quinazolin-4-yl]piperazine-1-carboxylic acid(4-isopropoxyphenyl)-amide, and has the structure of formula (I).

As used herein, the term “Form 1” is meant to describe Form 1 of the (L)-lactate salt of 4-[6-methoxy-7-(3-piperidin-1-yl-propoxy)quinazolin-4-yl]piperazine-1-carboxylic acid(4-isopropoxyphenyl)-amide.

As used herein, the term “Form 2” is meant to describe Form 2 of the (L)-lactate salt of 4-[6-methoxy-7-(3-piperidin-1-yl-propoxy)quinazolin-4-yl]piperazine-1-carboxylic acid(4-isopropoxyphenyl)-amide.

As used herein, the term “Form 3” is meant to describe Form 3 of the (L)-lactate salt of 4-[6-methoxy-7-(3-piperidin-1-yl-propoxy)quinazolin-4-yl]piperazine-1-carboxylic acid(4-isopropoxyphenyl)-amide.

As used herein, the term “Form 4” is meant to describe Form 4 of the (L)-lactate salt of 4-[6-methoxy-7-(3-piperidin-1-yl-propoxy)quinazolin-4-yl]piperazine-1-carboxylic acid(4-isopropoxyphenyl)-amide.

As used herein, the term “crystalline” refers to a solid having a highly regular chemical structure. In particular, a crystalline Lactate Salt may be produced as one or more crystalline forms of the Lactate Salt. For the purposes of this application, the term “polymorph”, and phrases “single crystalline form” and “crystalline form” are synonymous; they distinguish between crystals that have different properties (e.g., different XRPD patterns, different DSC scan results). Pseudo-polymorphs are typically different solvates of a material, and thus their properties differ from one another. For the purposes of the application, the pseudo-polymorphs are one sub-category of the term “polymorph”. Thus, each distinct polymorph or pseudo-polymorph of the Lactate Salt is considered to be a “single crystalline form” or “crystalline form” herein.

“Substantially crystalline” refers to the Lactate Salt that may be at least a particular weight percent crystalline. Particular weight percentages are 10%, 20%, 30%, 40%, 50%, 60%, 70%, 75%, 80%, 85%, 87%, 88%, 89%, 90%, 91%, 92%, 93%, 94%, 95%, 96%, 97%, 98%, 99%, 99.5%, 99.9%, or any percentage between 10% and 100%. In some embodiments, substantially crystalline refers to the Lactate Salt that is at least 70% crystalline. In other embodiments, substantially crystalline refers to the Lactate Salt that is at least 90% crystalline. In yet other embodiments, substantially crystalline refers to the Lactate Salt that is at least 95% crystalline.

As used herein, the term “solvate or solvated” means a physical association of a compound of this invention with one or more solvent molecules. This physical association includes hydrogen bonding. In certain instances the solvate will be capable of isolation, for example when one or more solvent molecules are incorporated in the crystal lattice of the crystalline solid. “Solvate or solvated” encompasses both solution-phase and isolable solvates. Representative solvates include, for example, a hydrate, ethanolates or a methanolate. The physical properties of a solvate typically differ from other solvates, and from unsolvated forms of the compound. Because the chemical composition also differs between solvates these forms are referred to as “pseudo-polymorphs”.

As used herein, the terms “hydrate or hydrated” is used to indicate a solvate wherein the solvent molecule is H₂O that is present in a defined stoichiometric amount, and may for example, include hemihydrate, monohydrate, dihydrate, or trihydrate. As used herein, the term “anhydrate” is a compound of the invention that contains no H₂O incorporated in its crystal lattice.

Unless otherwise explicitly stated, structures depicted herein are meant to include all hydrates, anhydrates, solvates and polymorphs thereof.

As used herein, the term “mixture” is used to refer to the combined elements of the mixture regardless of the phase-state of the combination (e.g., liquid or liquid/crystalline).

As used herein, the term “seeding” is used to refer to the addition of a crystalline material to initiate recrystallization or crystallization.

As used herein, the term “antisolvent” is used to refer to a solvent in which compounds of the invention are poorly soluble.

As used herein, the term “subject” is preferably a bird or mammal, such as a human, but can also be an animal in need of veterinary treatment, e.g., domestic animals (e.g., dogs, cats, and the like), farm animals (e.g., cows, sheep, fowl, pigs, horses, and the like) and laboratory animals (e.g., rats, mice, guinea pigs, and the like).

As used herein, the terms “treating” or “treatment” mean prevention, partial alleviation, or cure of a disease, disorder or condition. The compounds and compositions of this invention are useful in treating tyrosine kinase mediated diseases, disorders or conditions, particularly PDGFR, c-Kit or Flt-3, mediated diseases, disorders or conditions. Inhibiting tyrosine kinase activity may serve to treat a number of diseases, involving cell survival, proliferation, and migration, including cardiovascular disease (e.g. arteriosclerosis and vascular reobstruction), cancer (e.g. AML and malignant glioma), glomerulosclerosis, fibrotic disease and inflammation, as well as other cell-proliferative diseases.

As used herein, PDGFR-mediated disease, disorder or condition refers to a disease, disorder or condition in which the biological function of PDGFR affects the development and or course of the disease, disorder or condition, or in which modulation of PDGFR alters the development, course, and/or symptoms.

As used herein, Flt-3-mediated disease, disorder or condition refers to a disease, disorder or condition in which the biological function of Flt-3 affects the development and or course of the disease, disorder or condition, or in which modulation of Flt-3 alters the development, course, and/or symptoms.

As used herein, c-Kit-mediated disease, disorder or condition refers to a disease or condition in which the biological function of c-Kit affects the development and or course of the disease, disorder or condition, or in which modulation of c-Kit alters the development, course, and/or symptoms.

As used herein, the phrase “pharmaceutically effective amount” is meant to describe an amount of a compound, composition, medicament or other active ingredient effective in producing the desired therapeutic effect.

As used herein, the total weight of a single oral pharmaceutical dosage form, is determined by adding all the weights of the components in the oral pharmaceutical dosage form, and does not include the weight of any coatings which may be optionally applied to the oral pharmaceutical dosage form after it is formed. The total weight of a single oral pharmaceutical dosage form is used as the basis for calculating the weight percentage of each of the components of the oral pharmaceutical dosage form.

As used herein, the term “ribbon” is the resulting compaction sheet that is produced by passing a blend through a roller compactor.

As used herein, the term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%.

As used herein, the term “comprises” means “includes, but is not limited to.”

In one aspect, the present invention provides the (L)-lactate salt of 4-[6-methoxy-7-(3-piperidin-1-yl-propoxy)quinazolin-4-yl]piperazine-1-carboxylic acid(4-isopropoxyphenyl)-amide, or a crystalline form thereof. Accordingly, the present invention provides a compound of formula (I):

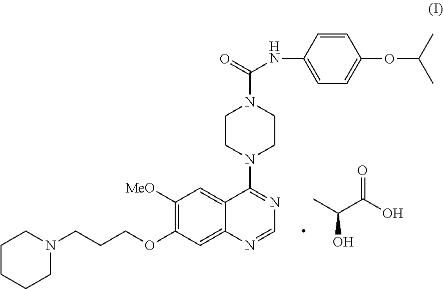

or a crystalline form thereof.

Provided herein is an assortment of characterizing information to describe the crystalline forms of the (L)-lactate salt of 4-[6-methoxy-7-(3-piperidin-1-yl-propoxy)quinazolin-4-yl]piperazine-1-carboxylic acid(4-isopropoxyphenyl)-amide (I). It should be understood, however, that not all such information is required for one skilled in the art to determine that such particular form is present in a given composition, but that the determination of a particular form can be achieved using any portion of the characterizing information that one skilled in the art would recognize as sufficient for establishing the presence of a particular form, e.g., even a single distinguishing peak can be sufficient for one skilled in the art to appreciate that such particular form is present.

Some of the crystalline forms of the Lactate Salt described herein exhibit considerably increased aqueous solubility over the compound of formula (II). For example, in water, the free base has a solubility of about 1.24 mg/mL at a pH of 6.95; Form 1 has a solubility of greater than 450 mg/mL at physiologically relevant pH's; and Form 4 has a solubility of greater than 325 mg/mL at physiologically relevant pH's.

In some embodiments, the Lactate Salt is substantially crystalline. Non-limiting examples of a crystalline Lactate Salt include a single crystalline form of the Lactate Salt or a mixture of different crystalline forms. An embodiment of the invention is also directed to a Lactate Salt that excludes one or more designated crystalline forms from a particular weight percentage of Lactate Salt. Particular weight percentages may be 10%, 20%, 30%, 40%, 50%, 60%, 70%, 75%, 80%, 85%, 87%, 88%, 89%, 90%, 91%, 92%, 93%, 94%, 95%, 96%, 97%, 98%, 99%, 99.5%, 99.9%, or any percentage between 10% and 100%.

Other embodiments of the invention are directed to the Lactate Salt being a designated crystalline form. The designated crystalline form may be a particular percentage by weight of the Lactate Salt. Particular weight percentages are 10%, 20%, 30%, 40%, 50%, 60%, 70%, 75%, 80%, 85%, 87%, 88%, 89%, 90%, 91%, 92%, 93%, 94%, 95%, 96%, 97%, 98%, 99%, 99.5%, 99.9%, or any percentage between 10% and 100%. In some embodiments, when a particular percentage by weight of a Lactate Salt is a designated crystalline form, the remainder of the Lactate Salt is a combination of amorphous Lactate Salt, and one or more crystalline forms of the Lactate Salt excluding the designated crystalline form. In some other embodiments, when a particular percentage by weight of a Lactate Salt is a designated crystalline form, the remainder of the Lactate Salt is amorphous Lactate Salt.

Examples of crystalline forms include the descriptions of crystalline forms characterized by one or more properties as discussed herein. The descriptions characterizing the crystalline forms may also be used to describe the mixture of different crystalline forms that may be present in a crystalline Lactate Salt.

In the following description of the Lactate Salt, embodiments of the invention may be described with reference to a particular crystalline “Form” of a Salt. However, the particular crystalline forms of each Salt may also be characterized by one or more of the characteristics of the polymorph as described herein, with or without regard to referencing a particular “Form”.

Form 1

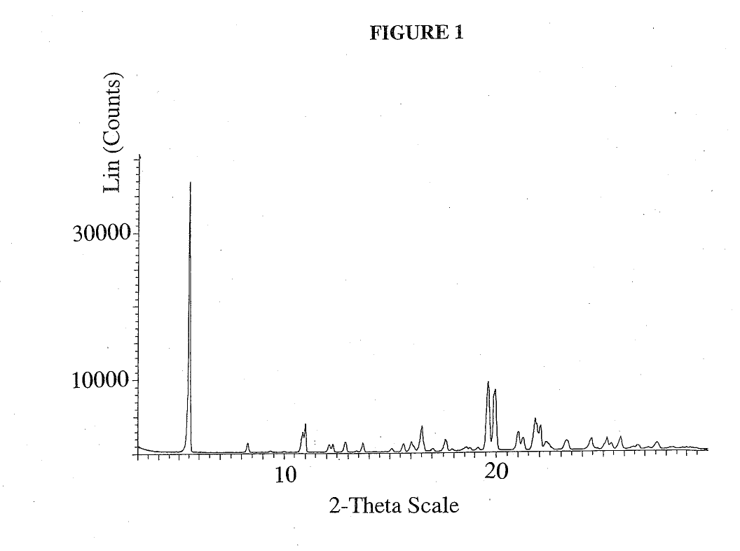

In one embodiment of the invention, a crystalline form, Form 1, of the Lactate Salt is characterized by the X-ray powder diffraction (XRPD) pattern shown in FIG. 1, and data shown in Table 1, obtained using CuKα radiation. In a particular embodiment of the invention, Form 1 can be characterized by one or more of the peaks taken from FIG. 1.

TABLE 1

| Angle | |
|---|---|
| 2-θ ° | Intensity % |
| 5.50 | 100.0 |
| 8.27 | 3.7 |
| 8.92 | 0.4 |
| 9.35 | 0.7 |
| 9.99 | 0.5 |
| 10.87 | 7.5 |
| 10.98 | 10.6 |
| 12.11 | 3.0 |
| 12.28 | 3.1 |
| 12.87 | 3.9 |
| 13.70 | 3.5 |
| 15.09 | 1.5 |
| 15.65 | 3.2 |
| 16.03 | 3.9 |
| 16.52 | 9.6 |
| 17.61 | 4.6 |
| 19.65 | 25.5 |
| 19.97 | 22.7 |
| 21.02 | 7.1 |
| 21.26 | 4.9 |
| 21.83 | 11.9 |
| 22.32 | 3.4 |

In another embodiment of the invention, the peaks are identified at 2θ angles of 5.50°, 10.98°, 19.65°, 19.97°, and 21.83°. In a further particular embodiment, the peaks are identified at 2θ angles of 5.50°, 19.65°, and 19.97°.

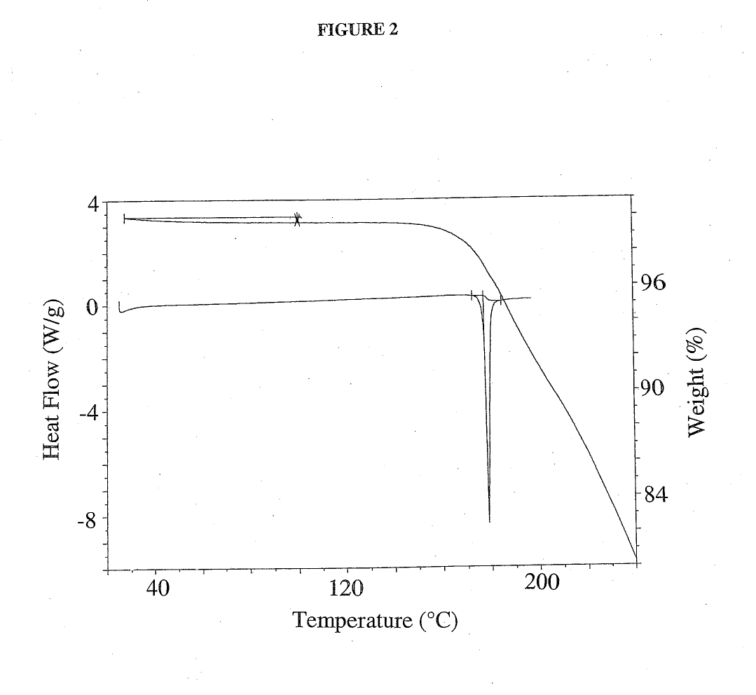

In another embodiment of the invention, Form 1 is characterized by the differential scanning calorimetry profile (DSC)/thermal gravimetric analysis (TGA) shown in FIG. 2. The DSC graph plots the heat flow as a function of temperature from a sample, the temperature rate change being about 10° C./min. The profile is characterized by an endothermic transition with an onset temperature of about 177.0° C., and a melt at about 178.7° C. These temperatures have an error of ±2° C.

The TGA profile, also shown in FIG. 2, graphs the percent loss of weight of the sample as a function of temperature, the temperature rate change being about 10° C./min. The weight loss represents a loss of about 0.3142% of the weight of the sample as the temperature is changed from 25° C. to 250° C.

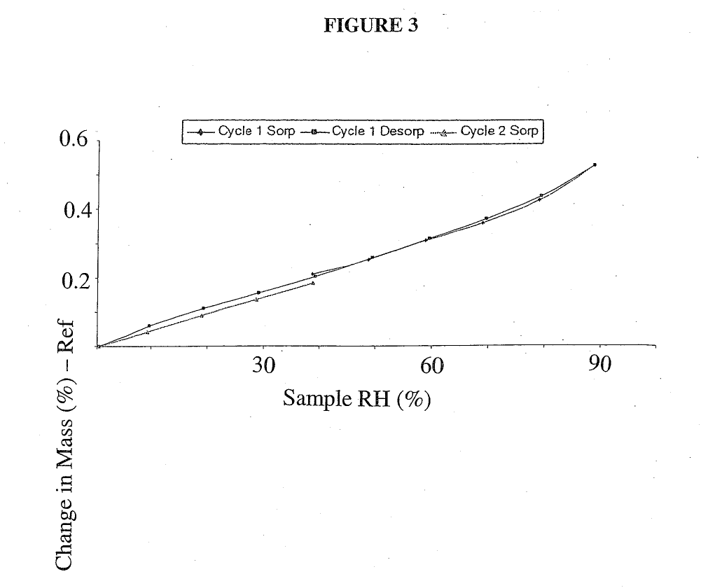

In another embodiment of the invention, Form 1 is characterized by the vapor sorption profiles (GVS), as shown in FIG. 3. The profile shows the change in weight of the sample as the relative humidity (RH) of the environment is changed by 10% RH intervals over a 0-90% RH range at a temperature of 25° C.

In another embodiment of the invention, Form 1 is characterized by at least one of the following features (I-i)-(I-iii):
(I-i) at least one of the X-ray powder diffraction peaks shown in Table 1;
(I-ii) an X-ray powder diffraction pattern substantially similar to FIG. 1; and
(I-iii) a differential scanning calorimetry (DSC) profile having an endothermic range of about 175° C. to about 185° C., with an onset temperature of about 177° C.

In a further embodiment of the invention, Form 1 is characterized by two of the features (I-i)-(I-iii). In another further embodiment of the invention, Form 1 is characterized by all of the features (I-i)-(I-iii).

Form 2

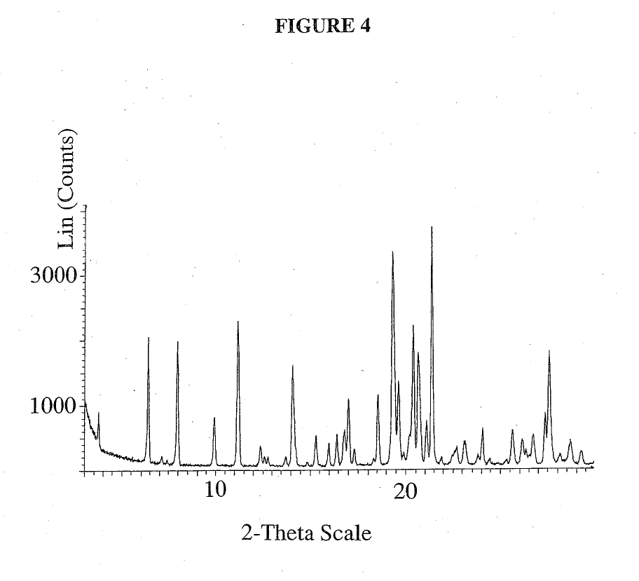

In one embodiment of the invention, a crystalline form, Form 2, of the Lactate Salt is characterized by the X-ray powder diffraction (XRPD) pattern shown in FIG. 4, and data shown in Table 2, obtained using CuKα radiation. In a particular embodiment of the invention, Form 2 can be characterized by one or more of the peaks taken from FIG. 4.

TABLE 2

| Angle | |
|---|---|
| 2-Theta ° | Intensity % |
| 6.38 | 56.3 |
| 7.12 | 5.9 |
| 7.98 | 53.5 |
| 9.92 | 21.6 |
| 11.19 | 61.9 |
| 12.37 | 9.6 |
| 12.59 | 5.5 |
| 12.78 | 5.2 |
| 13.73 | 5.5 |
| 14.12 | 44.3 |
| 15.31 | 14.0 |
| 16.00 | 10.3 |
| 16.43 | 14.9 |
| 16.81 | 16.0 |
| 17.03 | 28.9 |
| 17.32 | 8.6 |
| 18.57 | 31.1 |
| 19.39 | 92.1 |
| 19.65 | 36.9 |
| 20.41 | 60.6 |
| 20.68 | 48.6 |
| 21.10 | 20.5 |
| 21.44 | 100.0 |
| 22.73 | 9.6 |
| 23.15 | 11.1 |
| 24.09 | 17.1 |
| 25.65 | 15.1 |
| 26.17 | 12.4 |
| 26.75 | 14.2 |
| 27.43 | 23.4 |
| 27.65 | 47.6 |

In another embodiment of the invention, the peaks are identified at 2θ angles of 6.38°, 7.98°, 11.19°, 14.12°, 19.39°, 20.41°, 20.68°, 21.44°, and 27.65°. In a further particular embodiment, the peaks are identified at 2θ angles of 11.19°, 19.39°, 20.41°, and 21.44°.

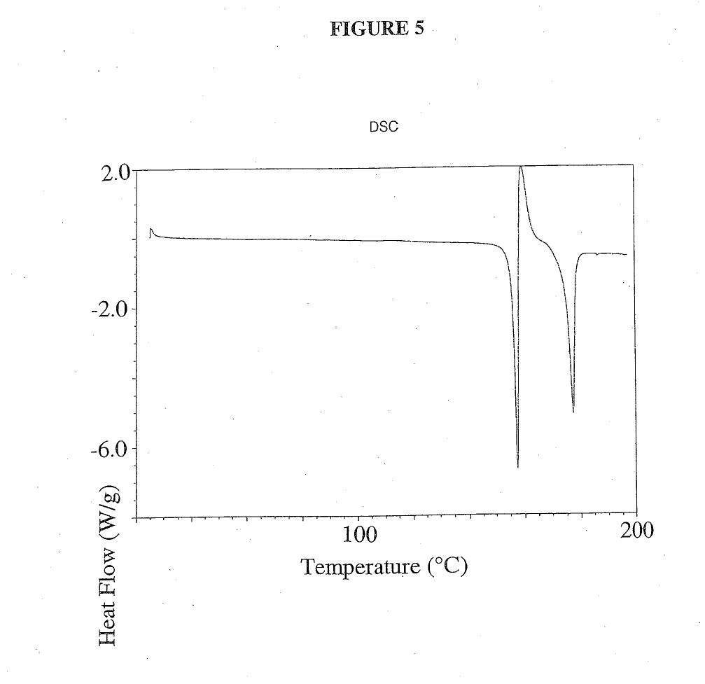

In another embodiment of the invention, Form 2 is characterized by the differential scanning calorimetry profile (DSC) shown in FIG. 5. The DSC graph plots the heat flow as a function of temperature from a sample, the temperature rate change being about 10° C./min. The profile is characterized by several endothermic and exothermic transitions. The first is an endothermic transition with an onset temperature of about 155.7° C., and a melt at about 157.4° C. (peak maximum). This is followed by an exothermic transition at about 159.2° C. (peak maximum). This is followed by a second endothermic transition with an onset temperature of about 174.5° C., and a melt at about 177.3° C. These temperatures have an error of ±2° C.

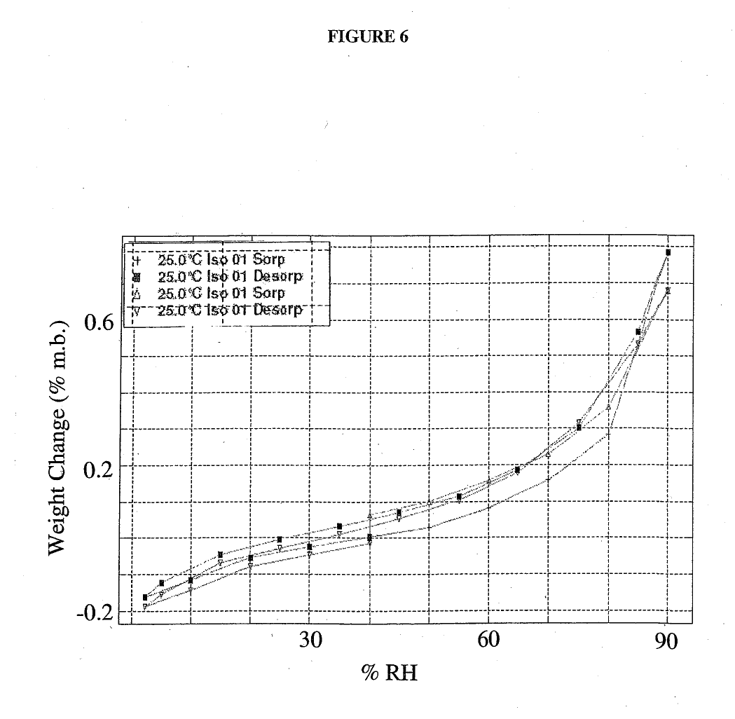

In another embodiment of the invention, Form 2 is characterized by the vapor sorption profiles (GVS), as shown in FIG. 6. The profile shows the change in weight of the sample as the relative humidity (RH) of the environment is changed by 10% RH intervals over a 0-90% RH range at a temperature of 25° C.

In another embodiment of the invention, Form 2 is characterized by at least one of the following features (II-i)-(II-iii):
(II-i) at least one of the X-ray powder diffraction peaks shown in Table 2;
(II-ii) an X-ray powder diffraction pattern substantially similar to FIG. 4; and
(II-iii) a differential scanning calorimetry (DSC) profile, comprising a first endothermic range of about 150° C. to about 160° C., with an onset temperature of about 155.7° C.

In a further embodiment of the invention, Form 2 is characterized by two of the features (II-i)-(II-iii). In a further embodiment of the invention, Form 2 is characterized by all of the features (II-i)-(II-iii).

Form 3

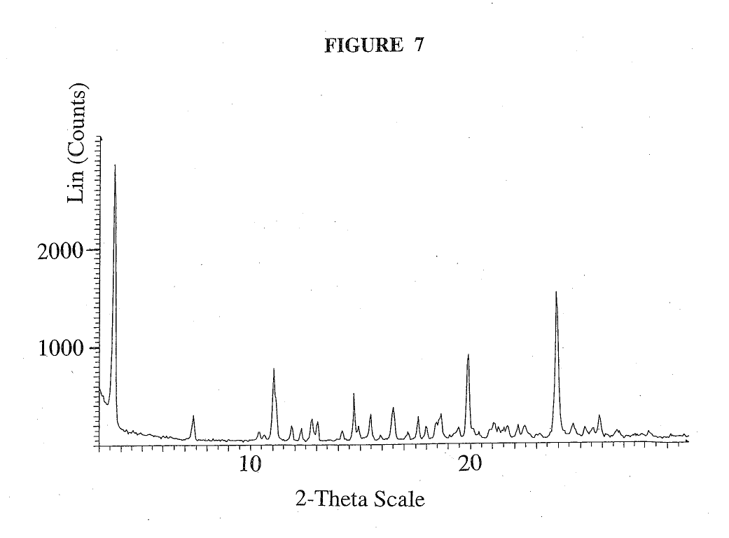

In one embodiment of the invention, a crystalline form, Form 3, of the Lactate Salt is characterized by the X-ray powder diffraction (XRPD) pattern shown in FIG. 7, and data shown in Table 3, obtained using CuKα radiation. In a particular embodiment of the invention, Form 3 can be characterized by one or more of the peaks taken from FIG. 7.

TABLE 3

| Angle | |
|---|---|
| 2-θ ° | Intensity % |
| 3.66 | 100.0 |
| 7.35 | 11.9 |
| 10.37 | 6.5 |
| 10.60 | 3.4 |
| 11.04 | 27.0 |
| 11.84 | 7.2 |
| 12.29 | 6.2 |
| 12.79 | 9.6 |
| 13.03 | 8.6 |
| 14.19 | 5.1 |
| 14.74 | 18.3 |
| 14.94 | 10.7 |
| 15.49 | 11.2 |
| 16.54 | 13.0 |
| 17.18 | 5.1 |
| 17.63 | 10.1 |
| 18.01 | 7.3 |
| 18.47 | 7.7 |
| 18.69 | 11.7 |
| 19.93 | 32.4 |
| 23.98 | 54.0 |

In another embodiment of the invention, the peaks are identified at 2θ angles of 3.66°, 11.04°, 19.93°, and 23.98°.

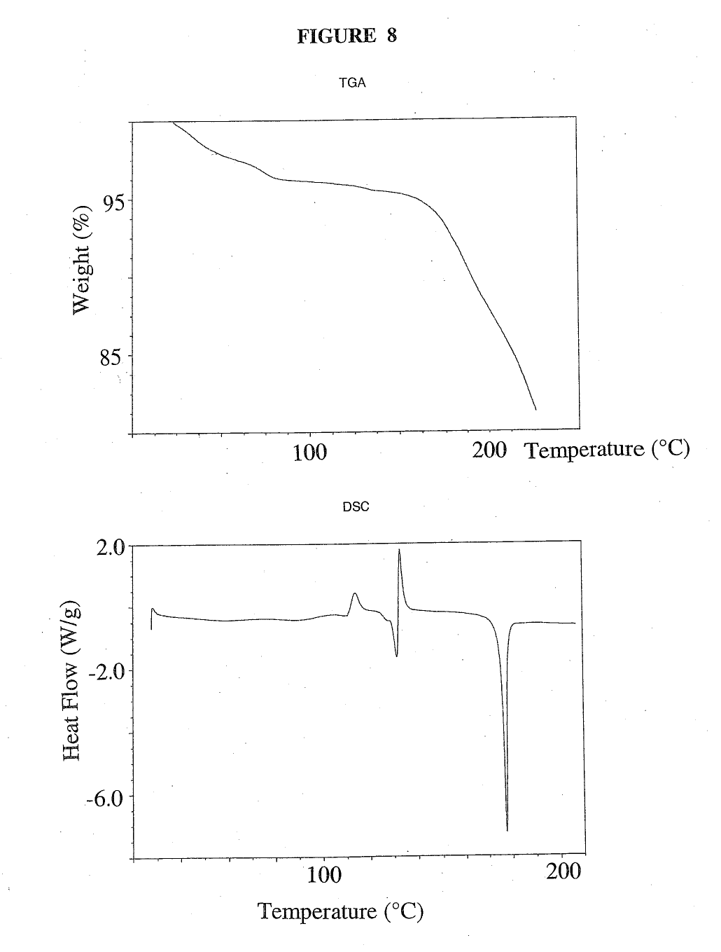

In another embodiment of the invention, Form 3 is characterized by the differential scanning calorimetry profile (DSC)/thermal gravimetric analysis (TGA) shown in FIG. 8. The DSC graph plots the heat flow as a function of temperature from a sample, the temperature rate change being about 10° C./min. The profile is characterized by several endothermic and exothermic transitions. The first is a weak endothermic transition with an onset temperature of about 110° C., which is quickly followed by an exothermic transition at about 114° C. (peak maximum). The second endothermic transition has an onset temperature of about 129° C., with a melt at about 131.5° C. (peak maximum), followed by an exothermic recrystallisation at about 133° C. (peak maximum). The third endothermic transition has an onset temperature of about 176° C. These temperatures have an error of ±2° C.

The TGA profile, also shown in FIG. 8, graphs the percent loss of weight of the sample as a function of temperature, the temperature rate change being about 10° C./min. The weight loss represents a loss of about 4.55% of the weight of the sample as the temperature is changed from 25° C. to 250° C. Karl Fischer measurements show a water content of about 4.8%, suggesting that the loss of weight seen in the TGA profile is due to the loss of water, indicating Form 3 is a hydrate.

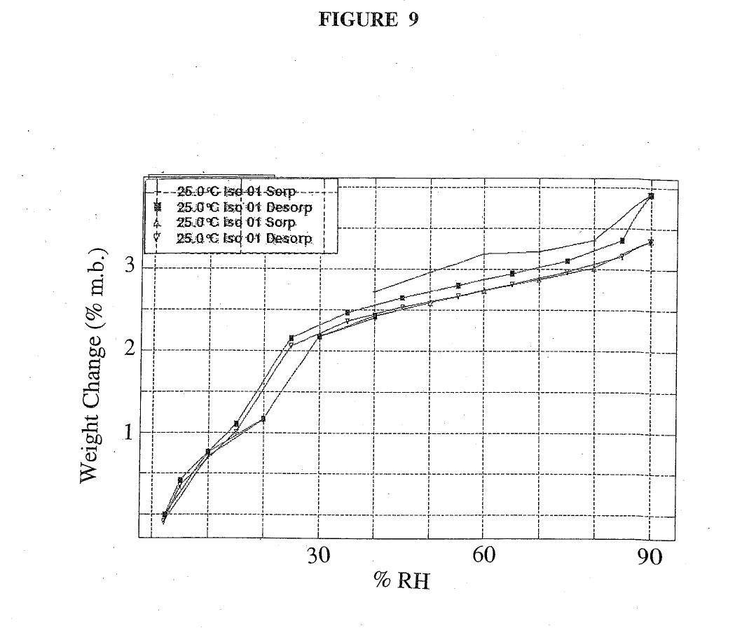

In another embodiment of the invention, Form 3 is characterized by the vapor sorption profile (GVS), as shown in FIG. 9. The profile shows the change in weight of the sample as the relative humidity (RH) of the environment is changed by 10% RH intervals over a 0-90% RH range at a temperature of 25° C.

In another embodiment of the invention, Form 3 is characterized by at least one of the following features (III-i)-(III-iii):
(III-i) at least one of the X-ray powder diffraction peaks shown in Table 3;
(III-ii) an X-ray powder diffraction pattern substantially similar to FIG. 7; and
(III-iii) a differential scanning calorimetry (DSC) profile substantially similar to FIG. 8.

In a further embodiment of the invention, Form 3 is characterized by two of the features (III-i)-(III-iii). In another further embodiment of the invention, Form 3 is characterized by all of the features (III-i)-(III-iii).

Form 4

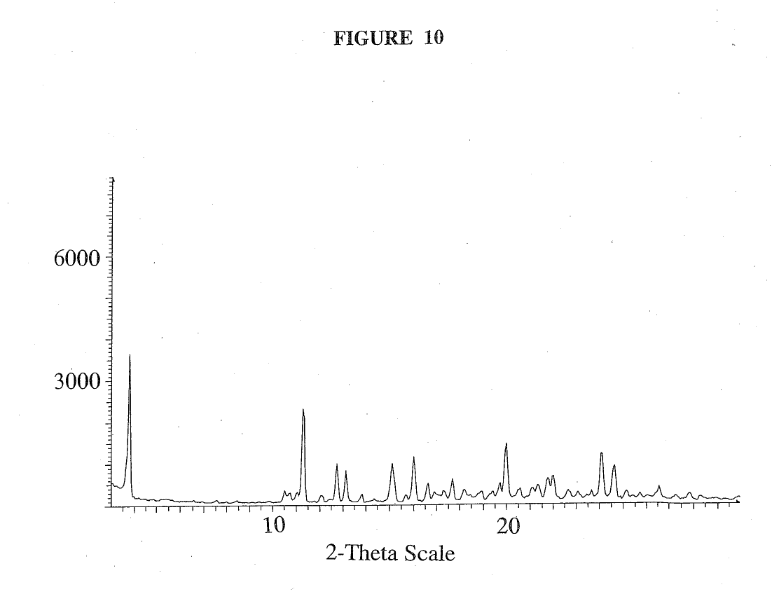

In one embodiment of the invention, a crystalline form, Form 4, of the Lactate Salt is characterized by the X-ray powder diffraction (XRPD) pattern shown in FIG. 10, and data shown in Table 4, obtained using CuKα radiation. In a particular embodiment of the invention, Form 4 can be characterized by one or more of the peaks taken from FIG. 10.

TABLE 4

| Angle | |
|---|---|
| 2-Theta ° | Intensity % |
| 3.76 | 100.0 |
| 10.53 | 7.8 |
| 10.73 | 8.9 |
| 11.04 | 8.5 |
| 11.30 | 63.3 |
| 12.08 | 5.9 |
| 12.71 | 26.8 |
| 13.11 | 17.6 |
| 13.82 | 6.8 |
| 15.15 | 26.7 |
| 15.71 | 6.0 |
| 16.02 | 31.1 |
| 16.58 | 12.3 |
| 16.89 | 9.1 |
| 17.31 | 8.6 |
| 17.67 | 16.7 |
| 18.19 | 9.1 |
| 18.88 | 7.9 |
| 19.39 | 7.8 |
| 19.74 | 13.5 |
| 20.03 | 39.7 |
| 20.55 | 8.7 |
| 21.14 | 10.3 |
| 21.36 | 11.3 |
| 21.78 | 16.4 |
| 21.98 | 17.2 |
| 24.15 | 31.9 |
| 24.66 | 24.7 |

In another embodiment of the invention, the peaks are identified at 2θ angles of 11.30°, 12.71°, 15.15°, 16.02°, 20.03°, 24.15°, and 24.66°. In a further particular embodiment, the peaks are identified at 2θ angles of 11.30°, 16.02°, 20.03°, and 24.15°.

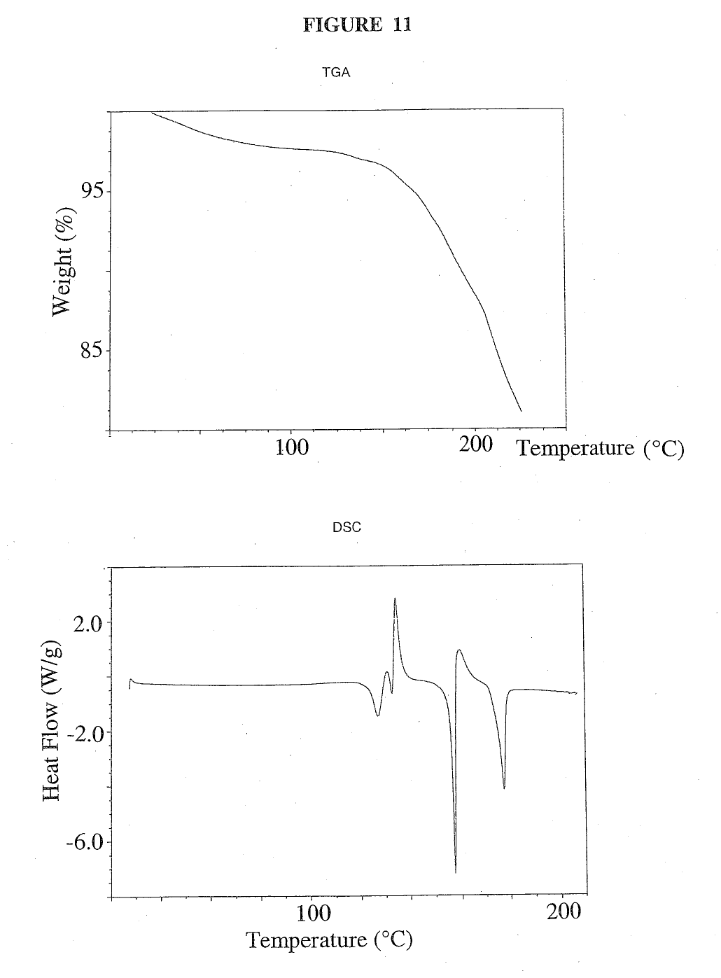

In another embodiment of the invention, Form 4 is characterized by the differential scanning calorimetry profile (DSC)/thermal gravimetric analysis (TGA) shown in FIG. 11. The DSC graph plots the heat flow as a function of temperature from a sample, the temperature rate change being about 10° C./min. The profile is characterized by several exothermic and endothermic transitions. The first is a weak endothermic transition with an onset temperature of about 123° C., which is followed by an exothermic recrystallisation at about 133° C. (peak maximum). The second endothermic transition has an onset temperature of about 155.7° C., followed by an exothermic recrystallisation at about 159.4° C. (peak maximum). The third transition has an onset temperature of about 174° C. These temperatures have an error of ±2° C.

The TGA profile also shown in FIG. 11, graphs the percent loss of weight of the sample as a function of temperature, the temperature rate change being about 10° C./min. The weight loss represents a loss of about 3.1% of the weight of the sample as the temperature is changed from 25° C. to 250° C. Karl Fischer measurements show a water content of about 2.5%, suggesting that the loss of weight is due to the loss of water, indicating Form 4 is a hydrate.

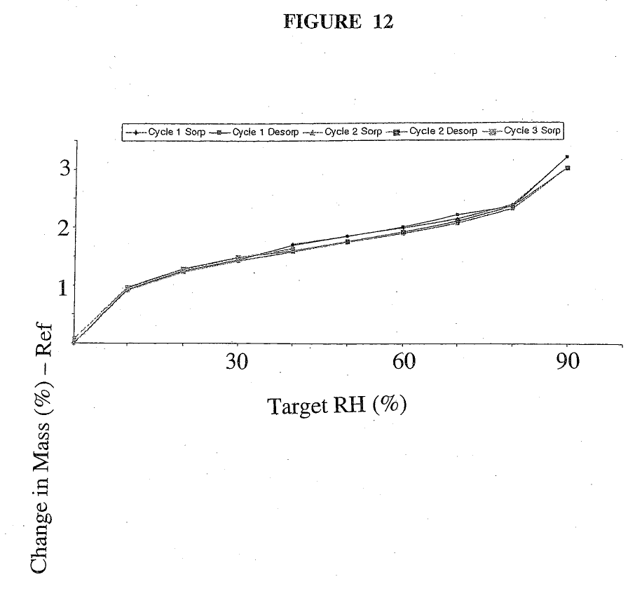

In another embodiment of the invention, Form 4 is characterized by the vapor sorption profiles (GVS), as shown in FIG. 12. The profile shows the change in weight of the sample as the relative humidity (RH) of the environment is changed by 10% RH intervals over a 0-90% RH range at a temperature of 25° C.

In another embodiment of the invention, Form 4 is characterized by at least one of the following features (IV-i)-(IV-iii):
(IV-i) at least one of the X-ray powder diffraction peaks shown in Table 4;
(IV-ii) an X-ray powder diffraction pattern substantially similar to FIG. 10;

and
(IV-iii) a differential scanning calorimetry (DSC) profile substantially similar to FIG. 11.

In a further embodiment of the invention, a single crystalline form of Form 4 is characterized by two of the features (IV-i)-(IV-iii). In a further embodiment of the invention, a single crystalline form of Form 4 is characterized by all of the features (IV-i)-(IV-iii).

Other embodiments of the invention are directed to a crystalline form of the Lactate Salt characterized by a combination of the aforementioned characteristics of any of the crystalline forms discussed herein. The characterization may be by any combination of one or more of the XRPD, TGA, DSC, and water sorption/desorption measurements described for a particular crystalline form. For example, a crystalline form of the Lactate Salt may be characterized by any combination of the XRPD results regarding the position of the major peaks in a XRPD scan; and/or any combination of one or more of the cell parameters derived from data obtained from a XRPD scan. A crystalline form of the Lactate Salt may also be characterized by TGA determinations of the weight loss associated with a sample over a designated temperature range; and/or the temperature at which a particular weight loss transition begins. DSC determinations of the temperature associated with the maximum heat flow during a heat flow transition and/or the temperature at which a sample begins to undergo a heat flow transition may also characterize the crystalline form. Weight change in a sample and/or change in sorption/desorption of water per molecule of anhydrous Lactate Salt as determined by water sorption/desorption measurements over a range of relative humidity (e.g., 0% to 90%) may also characterize a crystalline form of the Lactate Salt.

The combinations of characterizations that are discussed above may be used to describe any of the crystalline forms of the Lactate Salt discussed herein (e.g., Form 1, 2, 3 or 4).

Another aspect of the invention provides a process for the synthesis of the compound of Formula (II), or a hydrate thereof, as outlined in Schemes 1, 2 and 3 below. Scheme 1 describes steps (a) and (b).

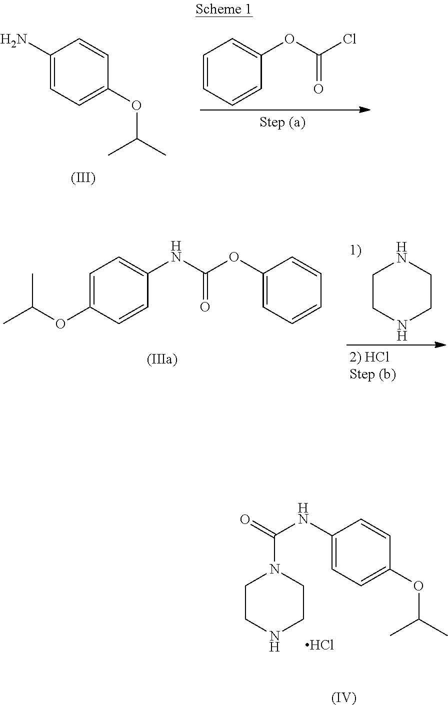

One embodiment of the invention describes the synthesis of a compound of formula (IIIa), or a salt thereof, from a compound of formula (III) in step (a) as shown in Scheme 1 above. Step (a) comprises treating a solution of the compound of formula (III) with a solution of phenyl chloroformate in the presence of a base, to generate an activated carbamate of formula (IIIa).

Suitable solvents for step (a) include, but are not limited to, acetonitrile, ethanol, isopropanol, sec-butanol, n-butanol, ethyl acetate, methylene chloride, chloroform, carbon tetrachloride, tetrahydrofuran, 2-methyltetrahydrofuran, isopropylacetate, dimethoxyethane, 1,4-dioxane, toluene, anisole, chlorobenzene, methyl tert-butyl ether, N,N′-dimethylformamide, N,N′-dimethylacetamide, N-methylpyrrolidinone, dimethylsulfoxide, diglyme, and mixtures thereof. In some embodiments, the solvent in step (a) is toluene, methylene chloride, 2-methyl tetrahydrofuran, ethyl acetate, isopropylacetate, chlorobenzene, acetonitrile, methyl tert-butyl ether, or mixtures thereof. In some embodiments, the solvent in step (a) to prepare the solution of compound (III) is acetonitrile. In some embodiments, the solvent in step (a) used to prepare the solution of phenyl chloroformate is toluene.

Suitable bases for step (a) are an alkaline earth metal base or an organic amine base. Examples of an alkaline earth metal base include, but are not limited to, potassium carbonate, sodium carbonate, calcium carbonate, lithium hydroxide, potassium hydroxide, sodium hydroxide, lithium hydrogen carbonate, potassium hydrogen carbonate, sodium hydrogen carbonate, lithium hydride, potassium hydride, sodium hydride, lithium tert-butoxide, potassium tert-butoxide, sodium tert-butoxide, cesium carbonate, and cesium hydroxide. Organic amine bases include, but are not limited to, trialkylamines, cyclic amines, pyridines, and substituted pyridines. Examples of these include, but are not limited to, triethylamine, pyridine, collidine, 2,6-lutidine, 4-dimethylaminopyridine, di-tertbutylpyridine, N-methylmorpholine, N-methylpiperidine, tetramethylguanidine, diazabicyclo[5.4.0]undec-7-ene (DBU), 1,4-diazabicyclo[2.2.2]octane, 1,5-diazabicycle[4.3.0]non-5-ene, N,N′-diisopropylethylamine, 1-azabicyclo[2.2.2]octane, tributylamine and trisopropylamine. In some embodiments, the base for step (a) is triethylamine.

The selection of an appropriate reaction temperature and reaction time for step (a) will depend largely on the base and solvent used. One skilled in the art will be able to select a suitable reaction temperature and reaction time in view of the reaction conditions being used. In some embodiments, the treating of step (a) is performed a temperature of about 0° C. to about 7° C.

In step (b), the compound of formula (IIIa) is treated with piperazine, followed by optional heating, followed by treatment of the resulting product with an acid to generate the compound of formula (IV), as a salt, as shown in Scheme 1 above. In some embodiments, the compound of formula (IIIa) and the piperazine are mixed together in a solvent, and the resulting mixture is optionally heated. In some other embodiments, a solution of the compound of formula (IIIa) is added slowly to piperazine, and the resulting mixture is optionally heated. In some other embodiments, a solution of the compound of formula (IIIa) is heated, and then a solution of piperazine is added slowly.

In some embodiments, the salt of the compound of formula (IV) is a hydrochloride. In some other embodiments, the salt of the compound of formula (IV) is hydrochloride, hydrobromide, hydroiodide, methanesulfonate, ethanesulfonate, p-toluenesulfonate, besylate, phosphate, sulfate, hydrogen sulfate, acetate, trifluoroacetate, propionate, citrate, maleate, fumarate, malonate, succinate, lactate, oxalate, tartrate or benzoate.

Suitable solvents for step (b) include, but are not limited to, methylene chloride, 2-methyl tetrahydrofuran, ethyl acetate, isopropylacetate, tetrahydrofuran, methanol, and isopropylalcohol. In some embodiments, the solvent in step (b) is ethyl acetate.

Step (b) is carried out at ambient temperature or an elevated temperature. One skilled in the art will be able to select a suitable reaction temperature and reaction time in view of the reaction conditions being used. In some embodiments, the heating of step (b) is carried out at a temperature of about 25° C. to about 80° C. In some other embodiments, the heating of step (b) is carried out at a temperature of about 35° C. to about 50° C. In some other embodiments, the heating of step (b) is carried out at a temperature of about 37° C. to about 42° C.

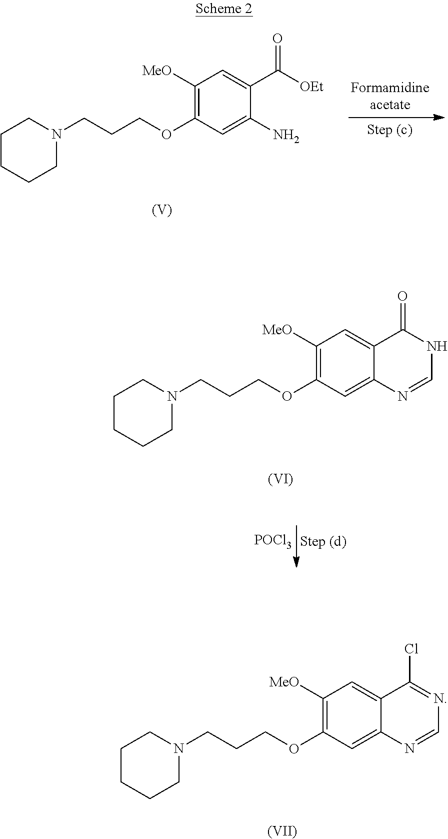

Another embodiment of the invention describes the synthesis of the compound of formula (VI) from the compound of formula (V) in step (c) as shown in Scheme 2 above. In step (c), a solution of the compound of formula (V) is treated with formamidine acetate, and optionally, a base. In some embodiments, in step (c), a base is added only during the work-up of the reaction.

Suitable solvents for step (c) include, but are not limited to, ethanol, isopropanol, sec-butanol, n-butanol, ethyl acetate, methylene chloride, chloroform, carbon tetrachloride, tetrahydrofuran, 2-methyltetrahydrofuran, dimethoxyethane, 1,4-dioxane, toluene, anisole, N,N′-dimethylformamide, N,N′-dimethylacetamide, N-methylpyrrolidinone, dimethylsulfoxide, diglyme, and mixtures thereof. In some embodiments, the solvent used in step (c) is N-methylpyrrolidinone.

Suitable bases for step (c) are an alkaline earth metal base or an organic amine base. Examples of an alkaline earth metal base include, but are not limited to, potassium carbonate, sodium carbonate, calcium carbonate, lithium hydroxide, potassium hydroxide, sodium hydroxide, lithium hydrogen carbonate, potassium hydrogen carbonate, sodium hydrogen carbonate, lithium hydride, potassium hydride, sodium hydride, lithium tert-butoxide, potassium tert-butoxide, sodium tert-butoxide, cesium carbonate, and cesium hydroxide. Organic amine bases include, but are not limited to, trialkylamines, cyclic amines, pyridines, and substituted pyridines. Examples of these include, but are not limited to, triethylamine, pyridine, collidine, 2,6-lutidine, 4-dimethylaminopyridine, di-tertbutylpyridine, N-methylmorpholine, N-methylpiperidine, tetramethylguanidine, diazabicyclo[5.4.0]undec-7-ene (DBU), 1,4-diazabicyclo[2.2.2]octane, 1,5-diazabicycle[4.3.0]non-5-ene, N,N′-diisopropylethylamine, 1-azabicyclo[2.2.2]octane, tributylamine and trisopropylamine. In some embodiments, the base used in step (c) is N,N′-diisopropylethylamine, pyridine, 1,4-diazabicyclo[2.2.2]octane, or collidine. In some embodiments, the base used in step (c) is N,N′-diisopropylethylamine.

Step (c) is preferably carried out at an elevated temperature. One skilled in the art will be able to select a suitable reaction temperature and reaction time in view of the reaction conditions being used. In some embodiments, step (c) is carried out at a temperature of about 50° C. to about 150° C. In some other embodiments, step (c) is carried out at a temperature of about 80° C. to about 150° C. In some other embodiments, step (c) is carried out at a temperature of about 115° C. to about 140° C. In certain embodiments, step (c) is carried out at a temperature of about 130° C.

Another embodiment of the invention describes the synthesis of the compound of formula (VII) from the compound of formula (VI) as shown above in Scheme 2. Step (d) comprises treating the compound of formula (VI) with POCl₃, and a base in a solvent to generate the compound of formula (VII).

In a particular embodiment, step (d) is conducted in a solvent mixture comprising an aromatic hydrocarbon solvent and a solvent that is a nitrile or ether. Suitable aromatic hydrocarbon solvents, include, but are not limited to, toluene, chlorobenzene, anisole, and mixtures thereof. Suitable nitrile solvents include, but are not limited to, acetonitrile. Suitable ether solvents include, but are not limited to, dimethyl ether, dimethoxyethane, tetrahydrofuran (THF), diethyl ether, 1,4-dioxane, and mixtures thereof. In another particular embodiment, the ratio of the aromatic hydrocarbon solvent to nitrile or ether solvent is from about 5:1 to about 4:1. In another particular embodiment, the solvent mixture comprises toluene and acetonitrile.

Suitable bases for step (d) are organic amine bases. Suitable organic amine bases include, but are not limited to, trialkylamines, cyclic amines, pyridines and substituted pyridines. Examples of these include, but are not limited to, triethylamine, pyridine, collidine, 2,6-lutidine, 4-dimethylaminopyridine, di-tertbutylpyridine, N-methylmorpholine, N-methylpiperidine, tetramethylguanidine, diazabicyclo[5.4.0]undec-7-ene (DBU), 1,4-diazabicyclo[2.2.2]octane, 1,5-diazabicycle[4.3.0]non-5-ene, N,N′-diisopropylethylamine, 1-azabicyclo[2.2.2]octane, tributylamine, and trisopropylamine. In some embodiments, the base in step (d) is N,N′-diisopropylethylamine.

Step (d) is preferably carried out at an elevated temperature. One skilled in the art will be able to select a suitable reaction temperature and reaction time in view of the reaction conditions being used. In some embodiments, step (d) is carried out at a temperature of about 20° C. to about 90° C. In some other embodiments, step (d) is carried out at a temperature of about 25° C. to about 60° C. In some other embodiments, step (d) is carried out at a temperature of about 35° C. to about 45° C. In certain embodiments, step (d) is carried out at a temperature of about 40° C. In certain other embodiments, step (d) is carried out at a temperature of about 80° C.

In certain embodiments, in step (d), the use of the solvent mixture of toluene and acetonitrile reduces the amounts of dimeric by-product formation, including dimers such as the compound of formula (VIa):

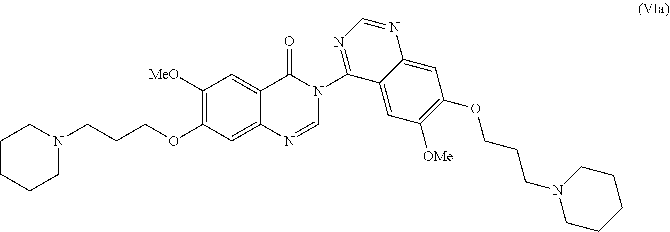 [SMILES: COc1cc2c(-n3cnc4cc(OCCCN5CCCCC5)c(OC)cc4c3=O)ncnc2cc1OCCCN1CCCCC1]

Further, by the use of the solvent mixture of toluene and acetonitrile in step (d), it is not necessary to use another solvent for the quench and work-up of the reaction. This significantly improves the process efficiency and simplicity for step (d).

In addition, there is no need to use a large excess of POCl₃ in step (d). In one embodiment, the amount of POCl₃ used is about 2.3 molar equivalents to about 0.5 molar equivalents relative to the amount of the compound of formula (VI). In another embodiment, the amount of POCl₃ is from about 1.8 molar equivalents to about 1.2 equivalents relative to the amount of the compound of formula (VI).

Another embodiment of the invention comprises the synthesis of the compound of formula (II), or a hydrate thereof, from the compound of formula (IV) and the compound of formula (VII) in step (e) as shown below in Scheme 3. Step (e) comprises the combining of the compounds of formula (IV) and formula (VII) in a solvent in the presence of a base.

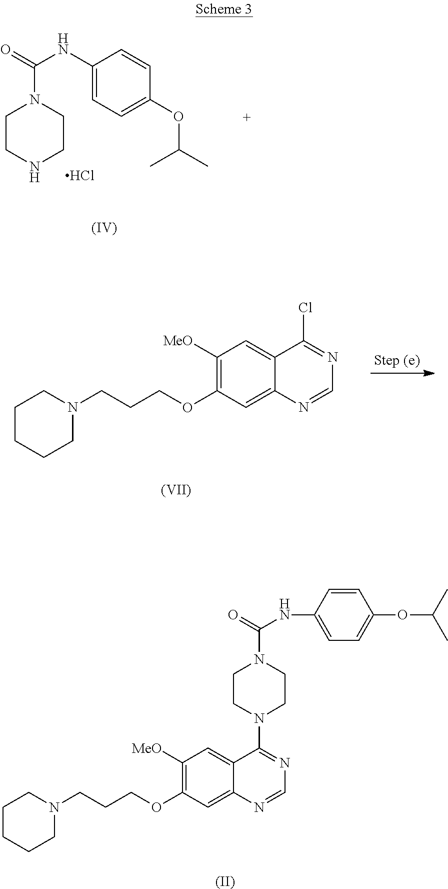

Suitable solvents for step (e) include, but are not limited to, isopropanol, sec-butanol, n-butanol, ethyl acetate, methylene chloride, chloroform, carbon tetrachloride, ethanol, tetrahydrofuran, 2-methyltetrahydrofuran, dimethoxyethane, 1,4-dioxane, toluene, anisole, N,N′-dimethylformamide, N,N′-dimethylacetamide, N-methylpyrrolidinone, dimethylsulfoxide, diglyme, and mixtures thereof. In some embodiments, the solvent in step (e) is ethanol.

Suitable bases for step (e) are an alkaline earth metal base or an organic amine base. Examples of an alkaline earth metal base include, but are not limited to, potassium carbonate, sodium carbonate, calcium carbonate, lithium hydroxide, potassium hydroxide, sodium hydroxide, lithium hydrogen carbonate, potassium hydrogen carbonate, sodium hydrogen carbonate, lithium hydride, potassium hydride, sodium hydride, lithium tert-butoxide, potassium tert-butoxide, sodium tert-butoxide, cesium carbonate, and cesium hydroxide. Organic amine bases include, but are not limited to, trialkylamines, cyclic amines, pyridines and substituted pyridines. Examples of these include, but are not limited to, triethylamine, pyridine, collidine, 2,6-lutidine, 4-dimethylaminopyridine, di-tertbutylpyridine, N-methylmorpholine, N-methylpiperidine, tetramethylguanidine, diazabicyclo[5.4.0]undec-7-ene (DBU), 1,4-diazabicyclo[2.2.2]octane, 1,5-diazabicycle[4.3.0]non-5-ene, N,N′-diisopropylethylamine, 1-azabicyclo[2.2.2]octane, tributylamine, and trisopropylamine. In some embodiments, the base used in step (e) is N,N′-diisopropylethylamine, triethylamine, potassium carbonate, cesium carbonate, or diazabicyclo[5.4.0]undec-7-ene (DBU). In some other embodiments, the base for step (e) is N,N′-diisopropylethylamine.

Step (e) is carried out at ambient temperature or an elevated temperature. One skilled in the art will be able to select a suitable reaction temperature and reaction time in view of the reaction conditions being used. In some embodiments, step (e) is carried out at a temperature of about 20° C. to about 50° C. In some other embodiments, step (e) is carried out at a temperature of about 20° C. to about 40° C. In some other embodiments, step (e) is carried out at a temperature of about 25° C.

In some embodiments, depending on the reaction conditions being used, step (e) produces the compound of formula (II) as a hydrate. In other embodiments, the compound of formula (II) is produced as a trihydrate. In other embodiments, the compound of formula (II) is dried to a particular percentage water content, as determined by Karl-Fischer analysis.

Another aspect of the invention provides processes for the synthesis of crystalline forms of the (L)-lactate salt of 4-[6-methoxy-7-(3-piperidin-1-yl-propoxy) quinazolin-4-yl]piperazine-1-carboxylic acid (4-isopropoxyphenyl)-amide of formula (I).

In one embodiment, the invention is directed to a process for producing Form 1 of the Lactate Salt from the compound of formula (II). In step 1, a solution of the compound of formula (II) in tetrahydrofuran is heated, and the water content is adjusted. This is followed by an optional polish filtering, and a second water content adjustment if necessary. Step 2 involves adding an 86% solution of (L)-lactic acid in water, followed by seeding with Form 1. The crystallization is continued in step 3, by adding isopropyl acetate as an anti-solvent, followed by a controlled cooling, after which the solids are collected, washed with isopropyl acetate and dried.

In some embodiments, in step 1 above, the solution of the compound of formula (II) in tetrahydrofuran is heated at a temperature of about 40° C., and the water content is adjusted to between about 0% to about 4%. In some other embodiments, in step 1 above, the solution of the compound of formula (II) in tetrahydrofuran is heated at a temperature of about 60° C., and the water content is adjusted to between about 0% to about 2%.

In some embodiments, in step 2 above, the solution is held at the temperature of step 1 for about two hours or less after the addition of the solution of (L)-lactic acid and Form 1 seed.

In some embodiments, in step 3 above, the isopropyl acetate is added over about 10 hours. In some other embodiments, in step 3 above, the controlled cooling is to a temperature of about 18° C. to about 24° C., over a period of about 6 hours to about 10 hours.

In another embodiment, Form 1 is produced by adding ethanol to the compound of formula (II), and heating the resulting mixture to about 55° C., after which an 85% solution of (L)-lactic acid in water is added. The solution is then cooled to about 40° C., and ethyl acetate is optionally added as an anti-solvent slowly over about three hours. Following cooling at 0° C., the suspension is filtered, and the solid dried.

In another embodiment, Form 1 of the Lactate Salt is produced by slurrying the amorphous Lactate Salt in a solvent. In such embodiments, suitable solvents are dioxane, toluene, cumene, methyl tert-butyl ether, tetralin, anisole, butyl actate, ethyl actate, isopropyl acetate, isopropanol, tetrahydrofuran, methyl ethyl ketone, acetonitrile, or nitromethane.

In another embodiment, Form 1 of the Lactate Salt is stirred in a solvent at room temperature, and no change in the crystalline form is observed. In such embodiments, suitable solvents are hexane, dioxane, toluene, cumene, methyl tert-butyl ether, anisole, ethyl acetate, isopropyl acetate, isopropyl alcohol, tetrahydrofuran, methyl ethyl ketone, acetone, ethanol, methanol, acetonitrile, nitromethane or N,N′-dimethylformamide

In another embodiment, Form 2 of the Lactate Salt is produced by slurrying the amorphous Lactate Salt in heptane. In one example, the slurry was kept in heat-cool cycles between room temperature and about 50° C., with four hours at each temperature for four days, resulting in the isolation of Form 2 of the Lactate Salt as determined by XRPD.

In another embodiment, Form 2 of the Lactate Salt is produced when Form 1 of the Lactate Salt is treated with a 1:1 solution of tetrahydrafuran:isopropyl acetate (THF:iPrOAc) with 2.5% water at room temperature, followed by seeding with either Form 2, or a mixture of Form 2 and Form 1. After stirring or shaking for between about 5 days and about 7 days, only Form 2 is isolated.

In another embodiment, Form 3 of the Lactate Salt is prepared when Form 1 of the Lactate Salt is treated with an optionally cooled 1:1 solution of THF:iPrOAc with 2.5% water at about 0° C., for between about 20 hours and about 90 hours. The resulting solid was analyzed by XRPD, and was found to be consistent with Form 3 of the Lactate Salt.

In another embodiment, Form 3 of the Lactate Salt is prepared by freeze-drying an aqueous solution of Form 1 of the Lactate Salt, followed by treatment of the lyophilized material with an optionally cooled 1:1 solution of THF:iPrOAc with 2.5% water at about 0° C., for between about 80 hours and about 90 hours. The resulting solid was analyzed by XRPD, and was found to be consistent with Form 3 of the Lactate Salt.

In another embodiment, Form 4 of the Lactate Salt is produced by treating the compound of formula (II) in tetrahydrofuran, and adjusting the water content to between about 3% and about 4% with a solution of 85% (L)-lactic acid in water at about 40° C. Seeding with Form 4, followed by addition of isopropyl acetate, then controlled cooling, leads to the isolation of Form 4.

In another embodiment, Form 4 of the Lactate Salt is produced by treating a solution of amorphous Lactate Salt in 1.2% water in 1:1 THF:iPrOAc at room temperature. The resulting solid is filtered and dried.

In another embodiment, heating any one of Form 2, Form 3 or Form 4 of the Lactate Salt to between about 135° C. and about 165° C. results in formation of Form 1 of the Lactate Salt.

In another embodiment, an aqueous solution of Form 1 is freeze-dried to produce the amorphous Lactate Salt.

Other embodiments of the invention are directed toward a pharmaceutical composition comprising a pharmaceutically acceptable carrier or diluent; and the Lactate Salt, or a crystalline form thereof. In other embodiments, the pharmaceutical composition comprises a pharmaceutically acceptable carrier or diluent, and a substantially crystalline Lactate Salt. In other embodiments, the pharmaceutical composition comprises a pharmaceutically acceptable carrier or diluent, and the Lactate Salt, wherein the Lactate Salt is at least 95% by weight a designated crystalline form; the crystalline forms being described herein. In other embodiments, the pharmaceutical composition comprises a pharmaceutically acceptable carrier or diluent, and the Lactate Salt, wherein the Lactate Salt is a single crystalline form; the single crystalline forms being described herein. In other embodiments, these compositions optionally further comprise one or more additional therapeutic agents.

As described above, the pharmaceutically acceptable compositions of the present invention additionally comprise a pharmaceutically acceptable carrier, which, as used herein, includes any and all solvents, diluents, or other liquid vehicle, dispersion or suspension aids, gelatin or polymeric capsule shell, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention. Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The Lactate Salt, or a crystalline form thereof, or a pharmaceutical composition thereof, can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol.

Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound. Alternatively, compositions for rectal or vaginal administration are gels or creams that can be prepared by mixing compounds with suitable non-irritating excipients such as oils or water to solubilize the compound and polymers and fatty alcohols can be added to thicken the formulation to increase the residual time in the rectal or vaginal cavity and release the active compound.

Solid dosage forms for oral administration include capsules, tablets, pills, powders, and granules. In such solid dosage forms, the active compound may optionally be mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, crospovidone, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, sodium stearyl fumarate, and mixtures thereof. In the case of capsules, tablets and pills, the dosage form may also comprise buffering agents or a flow aid such as colloidal silicon dioxide. In other embodiments, the active compound may be encapsulated in a gelatin or polymeric capsule shell without any additional agents (neat capsule shell).

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. The solid dosage forms may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

In some embodiments, the solid dosage form comprises the Lactate Salt, or a crystalline form thereof, and at least one of sodium stearyl fumarate, crospovidone, mannitol and colloidal silicon dioxide. In some embodiments, the solid dosage form comprises a tablet with a film coating. In some other embodiments, the solid dosage form comprises about 10% of a lubricant. In some other embodiments, the solid dosage form comprises about 9% of a disintegrant. In some embodiments, the solid dosage form has a high drug loading. In some embodiments, the solid dosage form comprises about 30% to about 60% by weight of the Lactate Salt, or a crystalline form thereof. In some embodiments, the solid dosage form comprises about 40% to about 50% by weight of the Lactate Salt, or a crystalline form thereof.

In one embodiment, the pharmaceutical composition comprises the compound of formula (I), wherein the compound of formula (I) is crystalline. In another embodiment, the compound of formula (I) in the pharmaceutical composition is at least about 95%, 96%, 97%, 98%, 99%, 99.5%, 99.9% of a designated crystalline form. In still yet another embodiment, the compound of formula (I) in the pharmaceutical composition is a designated crystalline form, wherein the crystalline form is Form 1.

In one embodiment, the pharmaceutical composition is an oral pharmaceutical dosage form. In another embodiment, the oral pharmaceutical dosage form is capsules, tablets, pills, powders, or granules. In yet another embodiment, the oral pharmaceutical dosage form is a tablet.

In one embodiment, the pharmaceutical composition comprises the compound of formula (I), or a crystalline form thereof, a lubricant, a disintegrant, a filler, and a glidant. In another embodiment, the pharmaceutical composition comprises about 30% to about 60% of the compound of formula (I), or a crystalline form thereof; about 6% to about 12% of a lubricant; about 6% to about 12% of a disintegrant; about 15% to about 50% of a filler; and about 0.3% to about 2% of a glidant, by weight as a percentage of total weight. In yet another embodiment, the pharmaceutical composition comprises about 45% to about 55% of the compound of formula (I), or a crystalline form thereof; about 9% to about 11% of a lubricant; about 8% to about 10% of a disintegrant; about 20% to about 40% of a filler; and about 0.8% to about 1.5% of a glidant, by weight as a percentage of total weight.

Suitable lubricants include, but are not limited to, magnesium stearate, glyceryl behenate, hydrogenated vegetable oil, talc, zinc stearate, calcium stearate, sucrose stearate, sodium stearyl fumarate, and mixtures thereof. In some embodiments, the lubricant is magnesium stearate, sodium stearyl fumarate, or mixtures thereof. In other embodiments, the lubricant is sodium stearyl fumarate.

In some embodiments, the pharmaceutical composition comprises a high level of lubricant. In some embodiments, the lubricant is present in an amount of about 2% to about 12%, by weight as a percentage of total weight. In some embodiments, the lubricant is present in an amount of about 6% to about 12%, by weight as a percentage of total weight. In some other embodiments, the lubricant is present in an amount of about 9% to about 11%, by weight as a percentage of total weight. In yet some other embodiments, the lubricant is present in an amount of about 6%, about 7%, about 8%, about 9%, about 10%, about 11%, or about 12%, by weight as a percentage of total weight. In still yet some other embodiments, the lubricant is present in an amount of about 9%, about 10%, or about 11%, by weight as a percentage of total weight. In some further embodiments, the lubricant is present in an amount of about 10%, by weight as a percentage of total weight.

In some embodiments, the lubricant comprises a first lubricant, which is intragranular, and a second lubricant, which is extragranular, which may be the same or different. In other embodiments, the first lubricant, and the second lubricant are the same. In some other embodiments, only the first lubricant is present. In yet some other embodiments, only the second lubricant is present.

In some embodiments, the first lubricant, and the second lubricant are each independently magnesium stearate, sodium stearyl fumarate, or mixtures thereof. In some other embodiments, the first lubricant, and the second lubricant are both sodium stearyl fumarate.

In some embodiments, the first lubricant and second lubricant are present in the same amount, provided that the total amount of lubricant is no greater than about 12%, by weight as a percentage of total weight. In other embodiments, the first lubricant and the second lubricant are present in different amounts, provided that the total amount of lubricant is no greater than about 12%, by weight as a percentage of total weight. In some embodiments, the first lubricant and second lubricant are each independently present in an amount of about 0% to about 12%, provided that the total amount of lubricant is no greater than about 12%, by weight as a percentage of total weight. In some other embodiments, the first lubricant and second lubricant are each independently present in an amount of about 2% to about 8%, provided that the total amount of lubricant is no greater than about 12%, by weight as a percentage of total weight. In yet some other embodiments, the first lubricant and second lubricant are each independently present in an amount of about 3% to about 6%, by weight as a percentage of total weight. In some further embodiments, the first lubricant and second lubricant are each independently present in an amount of about 3%, about 4%, about 5%, or about 6%, by weight as a percentage of total weight. In yet some further embodiments, the first lubricant and second lubricant are each independently present in an amount of about 5%, by weight as a percentage of total weight.

Suitable disintegrants include, but are not limited to, colloidal silicon dioxide, powdered cellulose, pregelatinized starch, calcium silicate, crospovidone, croscaramellose sodium, sodium lauryl sulfate, sodium starch glycolate, and mixtures thereof. In some embodiments, the disintegrant is crospovidone, calcium silicate, sodium starch glycolate, or mixtures thereof. In some other embodiments, the disintegrant is crospovidone.

In some embodiments, the pharmaceutical composition contains a high level of disintegrant. In some embodiments, the disintegrant is present in an amount of about 2% to about 12%, by weight as a percentage of total weight. In some embodiments, the disintegrant is present in an amount of about 6% to about 12%, by weight as a percentage of total weight. In some other embodiments, the disintegrant is present in an amount of about 8% to about 10%, by weight as a percentage of total weight. In yet some other embodiments, the disintegrant is present in an amount of about 6%, about 7%, about 8%, about 9%, about 10%, about 11%, or about 12%, by weight as a percentage of total weight. In still yet some other embodiments, the disintegrant is present in an amount of about 8%, about 9%, or about 10%, by weight as a percentage of total weight. In some further embodiments, the disintegrant is present in an amount of about 9%, by weight as a percentage of total weight.

In some embodiments, the disintegrant comprises a first disintegrant which is intragranular, and a second disintegrant, which is extragranular, which may be the same or different. In other embodiments, the first disintegrant, and the second disintegrant are the same. In some other embodiments, only the first disintegrant is present. In yet some other embodiments, only the second disintegrant is present.

In some embodiments, the first and second disintegrants are independently crospovidone, calcium silicate, sodium starch glycolate, or mixtures thereof. In some other embodiments, the first disintegrant, and the second disintegrant are both crospovidone.

In some embodiments, the first disintegrant and second disintegrant are each independently present in an amount of about 0% to about 12%, provided that the total amount of disintegrant is no greater than about 12%, by weight as a percentage of total weight. In some other embodiments, the first disintegrant and second disintegrant are each independently present in an amount of about 2% to about 8%, provided that the total amount of disintegrant is no greater than about 12%, by weight as a percentage of total weight. In yet some other embodiments, the first disintegrant and second disintegrant are each independently present in an amount of about 3% to about 6%, by weight as a percentage of total weight. In some further embodiments, the first disintegrant and second disintegrant are each independently present in an amount of about 3%, about 4%, about 5%, or about 6%, by weight as a percentage of total weight. In yet some further embodiments, the first disintegrant and second disintegrant are each independently present in an amount of about 4% to about 5%, by weight as a percentage of total weight.

The choice of filler was found to have an effect on the dissolution performance of the oral pharmaceutical dosage form. Suitable fillers include, but are not limited to, starch, sucrose, calcium phosphate, powdered cellulose, microcrystalline cellulose, silicified microcrystalline cellulose, isomalt, mannitol, lactose, and mixtures thereof. In some embodiments, the filler is microcrystalline cellulose, silicified microcrystalline cellulose, isomalt, mannitol, or mixtures thereof. In some other embodiments, the filler is mannitol. In some embodiments, the filler is present in an amount of about 10% to about 85.7%, by weight as a percentage of total weight. In some embodiments, the filler is present in an amount of about 10% to about 70%, by weight as a percentage of total weight. In some other embodiments, the filler is present in an amount of about 15% to about 50%, by weight as a percentage of total weight. In yet some other embodiments, the filler is present in an amount from about 20% to about 40%, by weight as a percentage of total weight. In some further embodiments, the filler is present in amount of about 28% to about 33%, by weight as a percentage of total weight. In yet some further embodiments, the filler is present in amount of about 28%, about 29%, about 30%, about 31%, about 32%, or about 33%, by weight as a percentage of total weight. In still yet some further embodiments, the filler is present in an amount of about 31.3%, by weight as a percentage of total weight.

In some embodiments, the filler comprises a first filler which is intragranular, and a second filler, which is extragranular, which may be the same or different. In other embodiments, the first filler, and the second filler are the same. In some other embodiments, only the first filler is present. In yet some other embodiments, only the second filler is present.

In some embodiments, the first filler and second filler are each independently present in an amount of about 0% to about 70%, provided that the total amount of filler is no greater than about 70%, by weight as a percentage of total weight. In some other embodiments, the first filler and second filler are each independently present in an amount of about 0% to about 50%, provided that the total amount of filler is no greater than about 50%, by weight as a percentage of total weight. In yet some other embodiments, the first filler and second filler are each independently present in an amount of about 5% to about 30%, provided that the total amount of filler is no greater than about 50%, by weight as a percentage of total weight. In still yet some other embodiments, the first filler and second filler are each independently present in an amount of about 10% to about 25%, by weight as a percentage of total weight.

Suitable glidants include, but are not limited to, silicon dioxide, colloidal silicon dioxide, talc, tribasic calcium phosphate, starch, magnesium trisilicate, powdered cellulose, and mixtures thereof. In some embodiments, the glidant is colloidal silicon dioxide. In some other embodiments, the glidant is present in an amount from about 0.3% to about 2%, by weight as a percentage of total weight. In yet some other embodiments, the glidant is present in an amount from about 0.8% to about 1.5%, by weight as a percentage of total weight. In still yet some other embodiments, the glidant is present in an amount of about 1%, by weight as a percentage of total weight.

In some embodiments, wherein the oral pharmaceutical dosage form is a tablet, the tablet is not film-coated. In some embodiments, wherein the oral pharmaceutical dosage form is a tablet, the tablet further comprises a film-coating. In some other embodiments, the film-coating system employed is Opadry® II (Colorcon, West Point, Pa.). In yet some other embodiments, the film-coating is present in an amount that is about a further 2% to about 4% of the total weight of the tablet. In still yet some other embodiments, the film-coating is present in an amount that is about a further 3% of the total weight of the tablet.

In some embodiments, the pharmaceutical composition comprises a high drug loading of the compound of formula (I), or a crystalline form thereof, as an active ingredient. In some embodiments, the compound of formula (I), or a crystalline form thereof, is present in an amount of about 10% to about 70%, by weight as a percentage of total weight. In some other embodiments, the compound of formula (I), or a crystalline form thereof, is present in an amount of about 30% to about 60%, by weight as a percentage of total weight. In yet some other embodiments, the compound of formula (I), or a crystalline form thereof, is present in an amount of about 45% to about 55%, by weight as a percentage of total weight.

In some embodiments, the pharmaceutical composition comprises about 30% to about 60% of the compound of formula (I), or a crystalline form thereof; about 6% to about 12% of sodium stearyl fumarate; about 6% to about 12% of crospovidone; about 15% to about 50% of mannitol; and about 0.3% to about 2% of colloidal silicon dioxide, by weight as a percentage of total weight. In another embodiment, the pharmaceutical composition comprises about 45% to about 55% of the compound of formula (I), or a crystalline form thereof; about 9% to about 11% of sodium stearyl fumarate; about 8% to about 10% of crospovidone; about 20% to about 40% of mannitol; and about 0.8% to about 1.5% of colloidal silicon dioxide, by weight as a percentage of total weight.

In another embodiment, the pharmaceutical composition comprises about 45% to about 55% of the compound of formula (I), Form 1; about 9% to about 11% of sodium stearyl fumarate; about 8% to about 10% of crospovidone; about 20% to about 40% of mannitol; and about 0.8% to about 1.5% of colloidal silicon dioxide, by weight as a percentage of total weight.

In some embodiments, the invention provides a process for the bulk production of an oral pharmaceutical dosage form of the compound of formula (I), or a crystalline form thereof, wherein the oral pharmaceutical dosage form is a tablet, comprising the steps of:
(a-1) blending the compound of formula (I), or a crystalline form thereof, and sieved first lubricant;
(a-2) blending the resulting mixture from step (a-1) with sieved first disintegrant, and sieved filler;
(a-3) sieving the resulting mixture from step (a-2), then further blending;
(a-4) roller compacting the resulting mixture from step (a-3) to a ribbon;
(a-5) milling the resulting ribbon from step (a-4);
(a-6) blending the resulting granules from step (a-5) with sieved glidant, and sieved second disintegrant;
(a-7) blending the resulting mixture from step (a-6) with sieved second lubricant;
(a-8) tableting the resulting mixture from step (a-7); and
(a-9) optionally film coating the resulting tablets from step (a-8).

In some embodiments, steps (a-4), and (a-5) can be omitted, the mixture resulting from step (a-3) is directly blended with sieved glidant and sieved second disintegrant in step (a-6). In some other embodiments, step (a-1), and step (a-2) are combined, and the resulting mixture is subjected to step (a-3). In yet some other embodiments, step (a-6), and step (a-7) are combined, and the resulting mixture is subjected to step (a-8).

In some other embodiments, the invention provides a process for the bulk production of an oral pharmaceutical dosage form of the compound of formula (I), or a crystalline form thereof, wherein the oral pharmaceutical dosage form is a tablet, comprising the steps of:
(b-1) blending the compound of formula (I), or a crystalline form thereof, and sieved sodium stearyl fumarate;
(b-2) blending the resulting mixture from step (b-1), with sieved crospovidone, and sieved mannitol;
(b-3) sieving the resulting mixture from step (b-2), then further blending;
(b-4) roller compacting the resulting mixture from step (b-3) to a ribbon;
(b-5) milling the resulting ribbon from step (b-4);
(b-6) blending the resulting granules from step (b-5) with sieved colloidal silicon dioxide, and sieved crospovidone;
(b-7) blending the resulting mixture from step (b-6) with sieved sodium stearyl fumarate;
(b-8) tableting the resulting mixture from step (b-7); and
(b-9) optionally film coating the resulting tablets from step (b-8).

In some embodiments, steps (b-4), and (b-5) can be omitted, the blend resulting from step (b-3) is directly blended with sieved colloidal silicon dioxide and sieved crospovidone in step (b-6). In some other embodiments, step (b-1), and step (b-2) are combined, and the resulting mixture is subjected to step (b-3). In yet some other embodiments, step (b-6), and step (b-7) are combined, and the resulting mixture is subjected to step (b-8).

In some embodiments, in steps (a-1) and (b-1), the particles size of the compound of formula (I), or a crystalline form thereof, is between about 10 and 1000 microns. In some other embodiments, in steps (a-1) and (b-1), the particle size of the compound of formula (I), or crystalline forms thereof, is between about 30 to about 400 microns. In some other embodiments, in steps (a-1) and (b-1), the particle size of the compound of formula (I), or crystalline forms thereof, is between about 50 to about 250 microns.

The process steps outlined above employ conventional apparatus or equipment. For a review, see e.g. Pharmaceutical Dosage Forms: Tablets: Unit Operations and Mechanical Properties, 1^{st} Ed., ed. L. L. Augsburger & S. W. Hoag, Taylor and Francis, Inc., 2008.

The blending steps outlined above can take place in any conventional blending or mixing apparatus. In some embodiments, the blending time for each individual blending step is between about 5 minutes and about 40 minutes. In some other embodiments, the blending time for each individual blending step is between about 5 minutes and about 15 minutes.

The milling of ribbon steps outlined above are performed using convential mesh screens. In some embodiments, the mesh screen size is about 0.8 mm to about 2.0 mm. In some other embodiments, the mesh screen size is about 0.8 mm, about 1.0 mm, about 1.25 mm, about 1.5 mm, about 1.75 mm, or about 2.0 mm.

The roller compacting steps above can take place in any conventional roller compactor apparatus. Suitable Roll Compression Forces in the range of about 0.5 kN/cm² to about 5 kN/cm² can be employed. It was found that an increase in the Roll Compression Force produced larger granules, which led to an increase in tablet strength and uniformity.

The pharmaceutical formulations of the invention exhibit improved processing behavior during tableting. The tableting steps above can take place in any conventional tablet press apparatus. It will be appreciated that the target compression force will vary depending on the size and shape of the tablets. In some embodiments, the target compression force during the tableting steps described above is between about 3 kN and about 30 kN. In some other embodiments, the target compression force during the tableting step is about 7.5 kN. In yet some other embodiments, the target compression force during the tableting step is about 13 kN.

In some embodiments, for the tableting steps described above, the resulting tablets are round in shape with a dual radius cap. In some other embodiments, for the tableting steps described above, the resulting tablets are modified capsule shaped tablets.

The optional film-coat step described above takes place using any conventional film-coating system. The film-coat is mixed with water and then sprayed in a perforated coating pan to coat the tablets. In other embodiments, the film-coating system employed is Opadry® II (Colorcon, West Point, Pa.).

The physical and chemical stability of the oral pharmaceutical dosage form may be tested in a conventional manner, for example, the measurement of dissolution, friability, disintegration time, assay for the compound of formula (I) degradation products, after storage at different temperatures for different lengths of time.

The pharmacological properties of the Lactate Salt, or a crystalline form thereof, or a pharmaceutical composition thereof, is such that it is suitable for use in the treatment of patients suffering from or subject to diseases, disorders or conditions mediated by tyrosine kinases, in particular PDGFR, c-Kit and Flt-3. Inhibiting tyrosine kinase activity, in particular PDGFR, c-Kit and Flt-3, may serve to treat a number of diseases involving cell survival, proliferation, and migration, including cardiovascular disease (e.g., arteriosclerosis and vascular reobstruction), cancer (e.g., AML and malignant glioma), glomerulosclerosis, fibrotic disease and inflammation, as well as other cell-proliferative diseases.

The present compounds are useful for treating or lessening the severity of a number of diseases involving cell survival, proliferation and migration. In some embodiments, these diseases and disorders include, but are not limited to, cardiovascular disease (e.g., arteriosclerosis and vascular reobstruction), glomerulosclerosis, fibrotic disease, and inflammation (Pandey et al., J. Med. Chem. 2002, 45:3772-3793).

In other embodiments, compounds of the invention are useful for treating cancer. The cancer types that may be treated include leukemia, such as acute myelogenous leukemia (AML) and brain tumor (Hegi et al., Annals of Oncology 2006, 17 (Supplement 10): x191-x197. The types of leukemias that may be treated include acute myelogenous leukemia (AML) (DeAngelo et al., Blood 2006, 108: 3674-3681). The types of brain tumor that may be treated include malignant gliomas, such as glioblastoma multiforme (GBM) (Kesari et al, Current Neurology and Neuroscience Reports 2005, 5:186-187; Rich and Bigner, Nature Reviews 2004, 3: 430-446).

In some embodiments, a method for treating cancer is provided comprising administering a pharmaceutically effective amount of the Lactate Salt, or a crystalline form thereof, or a pharmaceutical composition thereof, to a subject in need thereof.

In some embodiments, the Lactate Salt, or a crystalline form thereof, or a pharmaceutical composition thereof, is useful for treating AML. In some embodiments, the Lactate Salt, or a crystalline form thereof, or a pharmaceutical composition thereof, is useful for treating brain tumors. In some such embodiments, the Lactate Salt, or a crystalline form thereof, or a pharmaceutical composition thereof, is useful for treating malignant glioma. In certain embodiments, the Lactate Salt, or a crystalline form thereof, or a pharmaceutical composition thereof, is useful for treating glioblastoma multiforme.

The Lactate Salt, or a crystalline form thereof, or a pharmaceutical composition thereof, according to the methods of the present invention, may be administered using any amount and any route of administration effective for treating the disease. The exact amount required will vary from subject to subject, depending on the species, age, and general condition of the subject, the severity of the infection, the particular agent, its mode of administration, and the like. The Lactate Salt, or a crystalline form thereof, or a pharmaceutical composition thereof, are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disease being treated and the severity of the disease; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human. In certain embodiments, the compounds of the invention may be administered orally at dosage levels of about 0.01 mg/kg to about 50 mg/kg and preferably from about 1 mg/kg to about 25 mg/kg, of subject body weight per day, one or more times a day, to obtain the desired therapeutic effect.

The Lactate Salt, or a single crystalline form thereof, or pharmaceutical composition thereof, may be used in an application of monotherapy to treat a disorder, disease or symptom, it also may be used in combination therapy, in which the use of an inventive compound or composition (therapeutic agent) is combined with the use of one or more other therapeutic agents for treating the same and/or other types of disorders, symptoms and diseases. Combination therapy includes administration of the therapeutic agents concurrently or sequentially. Alternatively, the therapeutic agents can be combined into one composition which is administered to the patient.

In one embodiment, the Lactate Salt, or a crystalline form thereof, or a pharmaceutical composition thereof of the invention is used in combination with other therapeutic agents, such as other inhibitors of kinases, especially tyrosine kinases. In some embodiments, the Lactate Salt, or a crystalline form thereof, or a pharmaceutical composition thereof, of the invention is administered in conjunction with a therapeutic agent selected from the group consisting of cytotoxic agents, radiotherapy, and immunotherapy. It is understood that other combinations may be undertaken while remaining within the scope of the invention.

Another aspect of the invention relates to inhibiting PDGFR, c-Kit or Flt-3 activity in a biological sample or a patient, which method comprises administering to the patient, or contacting said biological sample with the Lactate Salt, or a crystalline form thereof, or a pharmaceutical composition. The term “biological sample”, as used herein, generally includes in vivo, in vitro, and ex vivo materials, and also includes, without limitation, cell cultures or extracts thereof; biopsied material obtained from a mammal or extracts thereof; and blood, saliva, urine, feces, semen, tears, or other body fluids or extracts thereof.

In order that this invention be more fully understood, the following preparative examples are set forth. These examples illustrate how to make or test specific compositions, and are not to be construed as limiting the scope of the invention in any way.

### EXAMPLES

### Abbreviations

DMF dimethylformamide
DMSO dimethylsulfoxide
EtOAc ethyl acetate
EtOH ethanol
iPrOAc isopropyl acetate
MeOH methanol
RO/DI reverse osmosis/deionized
THF tetrahydrofuran
hr hours
min minutes
m/z mass to charge
HPLC high-performance liquid chromatography
AUC area under curve
NMR nuclear magnetic resonance

Nuclear Magnetic Resonance (NMR):

Proton nuclear magnetic resonance spectra are obtained on a Varian Mercury 300 spectrometer at 300 MHz, on a Bruker AVANCE 300 spectrometer at 300 MHz, or on a Bruker AVANCE 500 spectrometer at 500 MHz.

X-Ray Powder Diffractometry (XRPD):

X-Ray Powder Diffraction patterns are collected on a Bruker D8 diffiactometer using Cu Kα radiation (40 kV, 40 mA), 0-20 goniometer, and divergence of V4 and receiving slits, a Ge monochromator and a Lynxeye detector. The software used for data collection is Diffrac Plus XRD Commander v2.5.0 and the data is analysed and presented using Diffrac Plus EVA v 11.0.0.2 or v 13.0.0.2.

Samples are run under ambient conditions as flat plate specimens. Approximately 10 mg of the sample is gently packed into a cavity cut into polished, zero-background (510) silicon wafer. The sample is rotated in its own plane during analysis. The details of the data collection are unless stated otherwise: (i) Angular range: 2 to 42 °2θ; (ii) Step size: 0.05 °2θ; and (iii) Collection time: 0.5 s·step⁻¹. Data for Form 1 are depicted in FIG. 1 and Table 1, data for Form 2 are depicted in FIG. 4 and Table 2; data for Form 3 are depicted in FIG. 7 and Table 3; and data for Form 4 are depicted in FIG. 10 and Table 4.

Differential Scanning Calorimetry (DSC):

DSC data are collected on a TA Instruments Q2000 equipped with a 50 position auto-sampler. The calibration for thermal capacity was carried out using sapphire and the calibration for energy and temperature was carried out using certified indium. Typically 0.5-3 mg of each sample, in a pin-holed aluminium pan, is heated at 10° C.·min⁻¹ from 25° C. to 250° C. A purge of dry nitrogen at 50 ml·min⁻¹ is maintained over the sample. Modulated temperature DSC is carried out using an underlying heating rate of 2° C.·min⁻¹ and temperature modulation parameters of ±0.2° C.·min⁻¹ and 40 seconds. The instrument control software was Advantage for Q Series v2.8.0.392 and Thermal Advantage v4.8.3, and the data is analysed using Universal Analysis v4.3A. Data for Form 1 are depicted in FIG. 2, data for Form 2 are depicted in FIG. 5, data for Form 3 are depicted in FIG. 8, and data for Form 4 are depicted in FIG. 11.

Thermal Gravimetric Analysis (TGA):

TGA data are collected on a TA Instruments Q500 TGA, equipped with a 16 position auto-sampler. The instrument is temperature calibrated using certified Alumel. Typically 5-30 mg of each sample is loaded onto a pre-tared platinum crucible, and aluminium DSC pan, and is heated at 10° C.·min⁻¹ from ambient temperature to 250° C. A nitrogen purge at 60 mL·min⁻¹ is maintained over the sample. The instrument control software is Advantage for Q Series v2.8.0.392 and Thermal Advantage v4.8.3. Data for Form 1 are depicted in FIG. 2, data for Form 3 are depicted in FIG. 8, and data for Form 4 are depicted in FIG. 11.

Gravimetric Vapor Sorption (GVS):

Gravimetric vapor sorption (GVS) data are collected using a either a Hiden IGASorp moisture sorption analyzer or a SMS DVS Intrinsic moisture sorption analyzer. 1) The Hiden IGASorp moisture sorption analyser is controlled by CFRSorp software. The sample temperature is maintained at 25° C. by a Huber re-circulating water bath. The humidity is controlled by mixing streams of dry and wet nitrogen, with a total flow rate of 250 ml·min⁻¹. The relative humidity is measured by a calibrated Vaisala RH probe (dynamic range of 0-95% RH), located near the sample. Typically 10-20 mg of sample is placed in a tared mesh stainless steel basket under ambient conditions. The sample is loaded and unloaded at 40% RH and 25° C. A standard moisture sorption isotherm is performed at 25° C. at 10% RH intervals over a 0-90% RH range. 2) The SMS DVS Intrinsic moisture sorption analyser is controlled by SMS Analysis Suite software. The sample temperature is maintained at 25° C. by the instrument controls. The humidity is controlled by mixing streams of dry and wet nitrogen, with a total flow rate of 200 ml·min⁻¹. The relative humidity is measured by a calibrated Rotronic probe (dynamic range of 1.0-100% RH), located near the sample. Typically 5-20 mg of sample is placed in a tared mesh stainless steel basket under ambient conditions. The sample is loaded and unloaded at 40% RH and 25° C. (typical room conditions). A standard moisture sorption isotherm is performed at 25° C. at 10% RH intervals over a 0-90% RH range. Data for Form 1 are depicted in FIG. 3, data for Form 2 are depicted in FIG. 6, data for Form 3 are depicted in FIG. 9, and data for Form 4 are depicted in FIG. 12.

Water Determination by Karl Fischer Titration:

The water content of a sample is measured on a Mettler Toledo DL39 Coulometer using Hydranal Coulomat AG reagent and an argon purge. Weighed solid samples are introduced into the vessel on a platinum TGA pan which is connected to a subaseal to avoid water ingress. Approx 10 mg of sample is used per titration and duplicate determinations are made.

### Example 1

### Preparation of phenyl 4-isopropoxyphenylcarbamate (IIIa)

Phenyl chloroformate (15.2 kg, 97.1 mol) was dissolved in toluene (115.7 kg) and cooled to 3° C. 4-isopropoxyaniline (III) (13.3 kg, 88.0 mol) was mixed with acetonitrile (43.4 kg) and slowly added to the phenyl chloroformate solution over 1 hour 40 minutes, followed by slow addition of triethylamine (9.8 kg, 96.8 mol) over 46 minutes. The mixture was heated to 17.5° C. and stirred for 3 hours 30 minutes until the reaction was deemed complete by HPLC. The product solution was washed with 1N HCl, followed by removing acetonitrile though an azeotropic distillation with additional toluene (52.3 kg). The solution was heated to 58° C. before slowly adding heptane (43.7 kg) over 1 hour 2 minutes, after which the slurry was cooled to 23.5° C. over 2 hours 35 minutes and stirred for an additional 2 hours 51 minutes. The material was isolated, washed twice with heptane, and dried at ≦40° C. for 3 hours 49 minutes. The product was discharged, yielding 19.1 kg of the title compound (IIIa) (80% yield, 100% purity HPLC AUC). ¹H NMR (300 MHz, CD₃OD, δ): 7.38 (m, 4H), 7.19 (m, 3H), 6.86 (d, J=9 Hz 2H), 4.52 (sept, J=6 Hz, 1H), 1.28 (d, J=6 Hz, 611).

### Example 2

### Preparation of N-(4-isopropoxyphenyl)piperazine-1-carboxamide hydrochloride (IV)

Phenyl 4-isopropoxyphenylcarbamate (IIIa) (19.1 kg, 70.4 mol) and piperazine (30.2 kg, 350.6 mol) were added to ethyl acetate (256.7 kg) and heated to 38.8° C. The reaction was stirred for 4 hours 15 minutes until deemed complete by HPLC. The reaction mixture was cooled to 23.3° C. and stirred for 18 hours 6 minutes before filtering to remove solids and washing the solids with ethyl acetate (15.5 kg). The filtrates were washed with a 10% aqueous brine solution, with the aqueous layer being back extracted with ethyl acetate (86.3 kg) and added to the organics. The organics were then washed twice more with a 10% brine solution. Slow addition of 1.25M HCl in isopropanol over 78 minutes followed by stirring for an additional 3 hours 56 minutes lead to the precipitation of the product. The solids were isolated, washed with isopropanol, and dried at ≦40° C. for 71 hours 37 minutes. The product was discharged, yielding 17.0 kg of the title compound (IV) (81% yield, 99.9% purity HPLC AUC). NMR (300 MHz, CD₃OD, δ): 7.23 (d, J=9 Hz, 2H), 6.83 (d, J=9 Hz, 2H), 4.52 (sept, J=6 Hz, 1H), 3.77 (m, 4H), 3.29 (m, 4H), 1.28 (d, J=6 Hz, 6H).

### Example 3

### Preparation of 6-methoxy-7-(3-(piperidin-1-yl)propoxy)quinazolin-4-ol (VI)

Ethyl 2-amino-5-methoxy-4-(3-(piperidin-1-yl)propoxy)benzoate (V) (1.0 kg, 2.97 mol) and formamidine acetate (0.464 kg, 4.46 mol) were added to 1-methyl-2-pyrrolidinone (5 L) and heated to 130° C. The mixture was allowed to stir for 6 hours until the reaction was deemed complete by HPLC. The product solution was cooled to 100° C., at which point N,N′-diisopropylethylamine (1.04 L, 5.94 mol) and RO/DI water (0.107 L) were added. The solution was allowed to stir for 90 minutes before cooling to 80° C. and seeding with 6-methoxy-7-(3-(piperidin-1-yl)propoxy)quinazolin-4-ol (0.01 kg, 0.003 mol). The product mixture was then cooled to 25° C. over 3 hours and stirred an additional 12 hours. The product was further crystallized by slowly adding acetonitrile (10 L) over 1 hour, stirring for 1 hour, slowly cooling to 5° C. over 1 hour, and stirring for 2 hours. The solids were isolated, washed twice with acetonitrile, and dried at ≦40° C. for 3 days. The product was discharged, yielding 0.822 kg of the title compound (87% yield, 99.74% purity HPLC AUC). ¹H NMR (300 MHz, d₆-DMSO, δ): 7.96 (s, 1H), 7.42 (s, 1H), 7.13 (s, 1H), 4.14 (m, 2H), 3.86 (s, 3H), 2.35 (m, 6H), 1.88 (m, 2H), 1.49 (m, 4H) and 1.30 (m, 2H).

### Example 4

### Preparation of 4-chloro-6-methoxy-7-(3-(piperidin-1-yl)propoxy)quinazoline (VII)

6-Methoxy-7-(3-(piperidin-1-yl)propoxy)quinazolin-4-ol (VI) (17.2 kg, 54.2 mol) was added to toluene (88.1 kg), then mixed with acetonitrile (20.5 kg) and N,N′-diisopropylethylamine (8.9 kg, 68.9 mol) before a controlled addition of phosphorous oxychloride (12.3 kg, 80.2 mol) over 9 minutes. The reaction solution was heated to 42° C. and stirred for 19 hours until the reaction was deemed complete by HPLC. The product solution was quenched by adding an ammonium hydroxide solution over 39 minutes. After the completion of the quench, the solution was at pH 11. The solution was then heated to 35° C., stirred for 47 minutes, and heated further to 57° C. The aqueous layer was removed and the organics were washed twice with RO/DI water. During the second water wash, an emulsion occurred. The emulsion layer was combined with the remaining organics and the two water washes were repeated. Lastly, the organics were washed with a saturated brine solution before undergoing a removal of toluene by an azeotropic distillation with additional acetonitrile (121.4 kg). The product was crystallized by first heating to 78° C. for complete dissolution, then cooling to 52° C., stirring for 1 hour 40 minutes, cooling to 25° C., stirring for 2 hours, cooling to 5° C., and finally, stirring for 2 hours 25 minutes. The solids were isolated, washed twice with cold acetonitrile (4.5° C.), and dried at ≦40° C. for 41 hours 40 minutes. The product was discharged, yielding 13.2 kg of the title compound (VII) (73% yield, 98.7% purity HPLC AUC). ¹H NMR (300 MHz, d₆-DMSO, δ): 7.96 (s, 1H), 7.42 (s, 1H), 7.13 (s, 1H), 4.14 (m, 2H), 3.86 (s, 3H), 2.35 (m, 6H), 1.88 (m, 2H), 1.49 (m, 4H) and 1.30 (m, 2H).

### Example 5

### Preparation of 4-[6-methoxy-7-(3-piperidin-1-yl-propoxy)quinazolin-4-yl]piperazine-1-carboxylic acid(4-isopropoxyphenyl)-amide (II) trihydrate

4-Chloro-6-methoxy-7-(3-(piperidin-1-yl)propoxy)quinazoline (VII) (12.9 kg, 38.4 mol), N-(4-isopropoxyphenyl)piperazine-1-carboxamide hydrochloride (IV) (12.2 kg, 40.7 mol), and diisopropylethylamine (12.9 kg, 99.8 mol) were added to ethanol (81.4 kg). The mixture was stirred for 21 hours and 54 minutes until the reaction was deemed complete by HPLC. The product mixture was heated to 49° C. and stirred for 35 minutes before undergoing a 10 μm polish filtration, with an ethanol (13.4 kg) rinse being added to the mixture. RO/DI water (75.9 L) was added before cooling the solution to 42° C. and seeding with 4-[6-methoxy-7-(3-piperidin-1-yl-propoxy)quinazolin-4-yl]piperazine-1-carboxylic acid(4-isopropoxyphenyl)-amide (II) trihydrate (13.04 g, 0.02 mol). After 4 hours 6 minutes of stirring, additional RO/DI water (53.1 L) was slowly added over 1 hour 36 minutes. The slurry was cooled to 22° C. over 5 hours 36 minutes, cooled further to 7° C. over an additional 94 minutes, and stirred for 2 hours 12 minutes. The solids were isolated, washed twice with a 4:5 ethanol:water solution, and dried at ≦30° C., then ≦40° C. for a total of 5 days 21 hours 21 minutes to a water content of 8% by Karl Fisher analysis. The product was discharged, yielding 21.3 kg of title compound (II) as a trihydrate. (90% yield, 97.1% purity HPLC AUC). ¹H NMR (300 MHz, d₆-DMSO, δ): 8.88 (s, 1H), 7.45 (s, 1H), 7.40 (s, 1H), 4.26 (m, 2H), 4.00 (s, 3H), 2.39 (m, 6H), 1.96 (m, 2H), 1.49 (m, 4H) and 1.39 (m, 2H).

### Example 6

### Preparation of 4-[6-methoxy-7-(3-piperidin-1-yl-propoxy)quinazolin-4-yl]piperazine-1-carboxylic acid(4-isopropoxyphenyl)-amide (L)-lactate (I) Form 1: Procedure 1

Before processing commenced, an 86% L-lactic acid solution was prepared by dissolving (L)-lactic acid (5.339 kg, 59.3 mol) in RO/DI water (0.835 L). 4-[6-Methoxy-7-(3-piperidin-1-yl-propoxy)quinazolin-4-yl]piperazine-1-carboxylic acid(4-isopropoxyphenyl)-amide (II) trihydrate (21.1 kg, 34.2 mol) was added to tetrahydrofuran (93.1 kg) and heated to 39° C. Water content of the solution was adjusted to 3.8% by adding RO/DI water (2.64 L) before polish filtering the solution though a 10 μm filter with a tetrahydrofuran (8.2 kg) rinse, after which the water content was readjusted to 3.8% by adding additional RO/DI water (0.342 L). The (L)-lactic acid solution (4.782 kg, 45.6 mol) was charged followed by seeding with Form 1 (211.8 g, 0.32 mol) and stirring for 65 minutes. The crystallization was continued by slowly adding isopropyl acetate (29.3 kg) over 4 hours 5 minutes, followed by additional isopropyl acetate (62.0 kg) over 5 hours 49 minutes. The crystallization was concluded with the following cooling profile: stirring for 62 minutes, cooling to 32° C. over 125 minutes, stirring for 60 minutes, cooling to 22° C. over 121 minutes, and stirring for 106 minutes. The solids were isolated, washed twice with isopropyl acetate, and dried at ≦45° C. for 3 days 19 hours 2 minutes. The product was discharged, yielding 20.2 kg of the title compound (90% yield, 99.5% purity HPLC AUC). ¹H NMR (300 MHz, MeOD, δ): 8.56 (s, 1H), 8.46 (s, 1H), 7.34 (d, J=9 Hz, 2H), 7.22 (s, 1H), 7.19 (s, 1H), 6.81 (d, J=9 Hz, 2H), 4.50 (sept, J=6 Hz, 1H), 4.17 (m, 2H), 3.93 (s, 3H), 3.67 (m, 8H), 2.38 (m, 6H), 1.94 (m, 2H), 1.49 (m, 4H) and 1.38 (m, 2H), 1.23 (d, J=6 Hz, 6H).

### Example 7

### Preparation of 4-[6-methoxy-7-(3-piperidin-1-yl-propoxy)quinazolin-4-yl]piperazine-1-carboxylic acid(4-isopropoxyphenyl)-amide (L)-lactate (I) Form 1: Procedure 2

4-[6-Methoxy-7-(3-piperidin-1-yl-propoxy)quinazolin-4-yl]piperazine-1-carboxylic acid(4-isopropoxyphenyl)-amide (II) trihydrate (250 g, 0.405 mol) was added to ethanol (750 ml) at room temperature. The mixture was warmed to 55° C. and an 85% (L)-Lactic acid in water solution (46 ml, 0.52 mol) was added. The solution was cooled to 40° C. and seeded with Form 1. After stirring at 40° C. for 1 hour 30 min, ethyl acetate (750 ml) was added over approximately 3 hours, the solution was then held at 40° C. for a further 2 hours, cooled to 0° C. over 7 hours, and held overnight at this temperature. The suspension was filtered, and the resulting solid dried to constant weight to yield 157 g (59% yield) of the title compound.

### Example 8

### Preparation of 4-[6-methoxy-7-(3-piperidin-1-yl-propoxy)quinazolin-4-yl]piperazine-1-carboxylic acid(4-isopropoxyphenyl)-amide (L)-lactate (I) Form 1: Procedure 3

To 4-[6-methoxy-7-(3-piperidin-1-yl-propoxy)quinazolin-4-yl]piperazine-1-carboxylic acid(4-isopropoxyphenyl)-amide (II) trihydrate (4.0 g, 6.49 mmol) was added THF (20 ml). The mixture was warmed to give a solution, and Karl Fischer analysis was performed. The water level was adjusted to 2.1%. At 60° C., an 85% (L)-Lactic acid in water solution (0.74 ml, 8.45 mmol) was added and the solution seeded with Form 1. The solution was held at 60° C. for 2 hours and isopropyl acetate (28 ml) was added over 10 hours. The suspension was cooled in 10° C. intervals over a total of 9 hours; then held at 20° C. for 2 hours when the suspension was filtered. The solid was washed with isopropyl acetate and dried to constant weight to yield 3.63 g (86% yield) of the title compound.

### Example 9

### Preparation of 4-[6-methoxy-7-(3-piperidin-1-yl-propoxy)quinazolin-4-yl]piperazine-1-carboxylic acid(4-isopropoxyphenyl)-amide (L)-lactate (I) Form 2: Procedure 1

107.8 mg of 4-[6-methoxy-7-(3-piperidin-1-yl-propoxy)quinazolin-4-yl]piperazine-1-carboxylic acid(4-isopropoxyphenyl)-amide (L)-lactate (I) Form 1 was dissolved in 200 μl of water in a 20 ml vial and Form 2 seed was added. 10 ml of iPrOAc:THF (dried over a 4 A molecular sieve) was added, and additional Form 2 seed added. After 1 hr only seed material was still observed in the suspension. 476.1 mg Form 1+20.2 mg Form 2 were then added to the suspension. The vial was then transferred to a shaker at room temperature. XRPD analysis after 1 day showed a mixture of Forms 1 and 2. After a further 4 days shaking at room temperature, XRPD analysis showed only Form 2. After drying under vacuum at room temp for 3 hours the yield of the title compound was 458 mg.

### Example 10

### Preparation of 4-[6-methoxy-7-(3-piperidin-1-yl-propoxy)quinazolin-4-yl]piperazine-1-carboxylic acid(4-isopropoxyphenyl)-amide (L)-lactate (I) Form 3: Procedure 1

600 mg of 4-[6-methoxy-7-(3-piperidin-1-yl-propoxy)quinazolin-4-yl]piperazine-1-carboxylic acid(4-isopropoxyphenyl)-amide (L)-lactate (I) Form 1 was pre-cooled to 0° C. before the addition of 6 ml of pre-cooled iPrOAc:THF (1:1)+2.5% water at 0° C. The suspension was stirred for 24 hrs at 0° C. After 24 hrs an aliquot was analyzed by XRPD which showed Form 3. The suspension was filtered under suction and the resulting solid was air dried overnight to give 543 mg of the title compound.

### Example 11

### Preparation of 4-[6-methoxy-7-(3-piperidin-1-yl-propoxy)quinazolin-4-yl]piperazine-1-carboxylic acid(4-isopropoxyphenyl)-amide (L)-lactate (I) Form 4: Procedure 1

To 4-[6-methoxy-7-(3-piperidin-1-yl-propoxy)quinazolin-4-yl]piperazine-1-carboxylic acid(4-isopropoxyphenyl)-amide (II) trihydrate (4.0 g, 6.49 mmol) was added THF (20 ml). The mixture was warmed to 40° C., and the water content adjusted to 3.2%. 85% (L)-Lactic acid in water (0.74 ml, 8.45 mmol) was added, and the solution was seeded with Form 4 (40 mg). Isopropyl acetate (20 ml) was added over 10 hours, the suspension was held at 40° C. for 1 hour, cooled to 30° C. over 2 hours, held at 30° C. for 1 hour, cooled to 20° C. over 2 hours, and held for a short time before the suspension was filtered, and the solid washed with isopropyl acetate. This yielded the title compound in a 90% yield.

### Example 12

### Form 1 Solubility

The equilibrium aqueous solubility of Form 1 was investigated under pH controlled conditions and the results are shown below in Table 5. The stability of the solubility solutions were evaluated and the parent peak purity was >99% after 12 days at ambient temperature in all samples tested by HPLC analysis.

TABLE 5
Form 1 Equilibrium Solubility Results

| | Solubility |
|---|---|
| pH | (mg/mL) |
| 3.51 | 496 |
| 3.97 | 465 |
| 4.23 | 511 |
| 5.96 | 458 |

### Example 13

### Form 4 Solubility

The equilibrium aqueous solubility of Form 4 was investigated under pH controlled conditions and the results are shown below in Table 6. The stability of the solubility solutions were evaluated and the parent peak purity was >99% after 12 days at ambient temperature in all samples tested by HPLC analysis.

TABLE 6
Form 4 Equilibrium Solubility Results

| | Solubility |
|---|---|
| pH | (mg/mL) |
| 3.65 | 359 |
| 5.85 | 412 |
| 6.03 | 327 |

### Example 14

### Kinase Phosphorylation Assays

The compounds of the invention are inhibitors of PDGFR, Flt-3 and c-Kit. Kinase phophorylation assays can be conducted as described in Pandey et al., J. Med. Chem. 2002, 45:3772-3793. The potent affinities for the kinases exhibited by the compounds of the invention can be measured as an IC₅₀ value (in nM), which is the concentration (in nM) of compound required to provide 50% inhibition of the kinase.

Other examples of assays that can be useful for evaluating and selecting a compound of the invention are described in WO 02/016351, U.S. Ser. No. 05/101,609 and U.S. Ser. No. 05/288,297 and U.S. Pat. No. 6,982,266 and below.

### Example 15

### Analysis of PDGFR-Beta Phosphorylation In Vitro

Approximately 5×10⁶ cells are plated into 10 cm Petri dishes, or 1×10⁶ cells are seeded into each well of a 6-well plate. Cells are incubated overnight at 37° C. Prior to use, cells are washed twice with PBS, are serum starved for 4 hours, and are pre-treated with 0-100 μM compound for 1 h prior to stimulation with 50 ng/mL PDGF-BB (Cell Signaling, Beverly, Mass.) for 5 min. PDGF stimulation is terminated with ice-cold PBS and total protein lysates are extracted with MPER buffer (Pierce) supplemented with 1 mM PMSF, 1 mM Na₃VO₄, 1 mM NaF, 1 μg/mL leupeptin, 1 μg/mL aprotinin, and 1 μg/mL pepstatin (Upstate, Charlottesville, Va.). Total protein lysates are processed, separated on gels, transferred to PVDF Immobilon-FL transfer membranes (Millipore, cat#IPFL00010) and immunoblotted with anti-phospho-PDGFR-beta (Y751) (Cell Signaling cat#3161L), anti-phospho-PDGFR-beta (Y857) (Santa Cruz Biotech, cat#sc-12907-R), and anti-total PDGFR-beta (Santa Cruz Biotech, cat#sc-432). To determine the percent of phosphor-PDGFR-beta inhibition, total and phospho-PDGFR-beta levels are quantified against linear standards using the Odyssey Infrared Imaging System (LI-COR Biosciences, Lincoln, Nebr.) and compared against the level obtained from samples taken at time 0.

### Example 16

### Inhibition of PDGFR-Beta Autophosphorylation in Tumor Xenografts

C6 rat glioma cells (ATCC, Rockville Md.) are grown in F12K (Kaighn's modification) medium (Gibco, Grand Island N.Y.) supplemented with 15% horse serum (GIBCO), 2.5% FBS (Hyclone, Logan Utah) 1.5 g/L sodium bicarbonate and 2 mM L-glutamine, and are maintained at 37° C. in 5% CO2. Four to nine week old NCr nu/nu immunocompromised mice (Taconic, Germanstown, N.Y.) are injected subcutaneously in the right flank with 1×10⁶ C6 glioma cells suspended in 0.1 mL Hank's balanced salt solution (GIBCO). Tumor size is assessed every 2-3 days using calipers, and mean tumor volume is calculated using the formula V=LW²/2. Mice with tumors around 400 mm³ are randomized into groups of 3-5 animals/group for pharmacokinetic/pharmacodynamic experiments. Animals receive a single dose of compound, are euthanized, the tumors are removed and stored frozen at −80° C., and blood is collected at a series of timepoints. Blood is collected into tubes with EDTA (Microtainer), and samples are spun at 10,000 rpm for 10 minutes. The plasma (the clear upper layer) is transferred to a separate tube and stored at −80° C. Plasma concentrations of the compound are measured using robust LC/MS/MS methods. Tumor samples may be analyzed for phosphorylation of PDGFR-beta using a quantitative western blotting procedure. For example, tumor homogenates are prepared using a Covaris sonicator. Tumor samples are pulverized and lysed in an appropriate buffer like the M-PER lysis buffer (Pierce), and are then homogenized using the Covaris sonicator. Lysates are run on 7% Tris-Acetate gel (Invitrogen) and blotted onto PVDF Immobilon-FL transfer membranes (Millipore, cat#IPFL00010). Membranes are immunoblotted with anti-phospho-PDGFR-beta (Y751) (Cell Signaling cat#3161L), anti-phospho-PDGFR-beta (Y857) (Santa Cruz. Biotech, cat#sc-12907-R), and anti-total PDGFR-beta (Santa Cruz Biotech, cat#sc-432). To determine the percent of phosphor-PDGFR-beta inhibition, total and phospho-PDGFR-beta levels are quantified against linear standards using the Odyssey Infrared Imaging System (LI-COR Biosciences, Lincoln, Nebr.) and compared against the level obtained from samples taken at time 0.

### Example 17

### Glioblastoma Tumor Xenograft Model

C6 rat glioma cells (1×10⁶) are suspended in 0.1 mL Hank's balanced salt solution (GIBCO), and injected subcutaneously into the right flank of 4 to 9 week-old NCr nu/nu immuno-compromised mice (Taconic, Germanstown, N.Y.). Tumor size is assessed every 2-3 days using calipers, and mean tumor volume is calculated using the formula V=LW²/2. Mice with tumors around 200 mm³ are randomized into groups of 10 animals for efficacy studies. The test compound is prepared in a vehicle such as 5% dextrose and is given by oral gavage on a daily or twice daily schedule, or can be given by subcutaneous injection daily or twice daily. Several different dose levels may be tested in a single experiment. Tumor growth inhibition is calculated using the formula TGI %=100(V_{c}−Vₜ)/V_{c}, where V_{c} and Vₜ are the mean tumor volume of the control and treated tumors on the last day of treatment, respectively.

### Example 18

### Preparation of 4-[6-methoxy-7-(3-piperidin-1-yl-propoxy)quinazolin-4-yl]piperazine-1-carboxylic acid(4-isopropoxyphenyl)-amide (L)-lactate (I) Form 1: Procedure 4

4-[6-Methoxy-7-(3-piperidin-1-yl-propoxy)quinazolin-4-yl]piperazine-1-carboxylic acid(4-isopropoxyphenyl)-amide (II) trihydrate (600 g, 0.973 mol) was added to ethanol (2400 ml) at room temperature. The mixture was stirred at ambient temperature for 30 min. Charcoal (60 g, 5 mol) was added and the mixture heated at 60° C. for 1 hour. The mixture was filtered through celite and the celite washed with ethanol (300 ml). The ethanolic mixture was transferred to a jacketed reactor, the first vessel was rinsed with ethanol, and further ethanol was added (total additional ethanol added approximately 1500 ml) followed by 85% (L)-Lactic acid in water (100 ml, 1.14 mol). The mixture was warmed to 70° C. and transferred to another jacketed reactor. The solution was cooled to 57° C. and seeded with Form 1. After 1 hour with the jacket temperature set at 60° C., ethyl acetate (6000 ml) was slowly added. Once the addition was complete the suspension was stirred for 1 hour, cooled to 20° C. over 5 hours, and then stirred at 20° C. overnight. The suspension was filtered, washed with ethyl acetate and dried in a vacuum oven at room temperature for approximately 40 hours to constant weight to yield 419 g (64% yield) of the title compound.

### Example 19

### Preparation of 4-[6-methoxy-7-(3-piperidin-1-yl-propoxy)quinazolin-4-yl]piperazine-1-carboxylic acid(4-isopropoxyphenyl)-amide (L)-lactate (I) Form 2: Procedure 2

Approximately 500 mg of 4-[6-methoxy-7-(3-piperidin-1-yl-propoxy)quinazolin-4-yl]piperazine-1-carboxylic acid(4-isopropoxyphenyl)-amide (L)-lactate (I) Form 1 was placed in a glass vial and 5 ml of a iPrOAc:THF mixture (1:1) was added. The suspension was slurried under ambient conditions for 1 hour before approximately 50 mg of 4-[6-methoxy-7-(3-piperidin-1-yl-propoxy)quinazolin-4-yl]piperazine-1-carboxylic acid(4-isopropoxyphenyl)-amide (L)-lactate (I) Form 2 (contaminated with a small amount of Form 1) was added. The sample was then cooled to 5° C. and held at this temperature for 3 days. XRPD analysis after 1 and 3 days showed a mixture of Forms 1 and 2. Stirring was maintained throughout. The sample was then brought up to room temperature and put in the shaker. XRPD analysis after an additional 3 days showed a mixture of Forms 1 and 2. 1% water was then added to the suspension which was then slurried for one more day. XRPD analysis of a small aliquot showed the material was Form 2. The rest of the suspension was filtered through a 5 micron Whatman filter cup. The material was dried at 25° C. for 18 hours under vacuum to give 400 mg of Form 2.

### Example 20

### Preparation of 4-[6-methoxy-7-(3-piperidin-1-yl-propoxy)quinazolin-4-yl]piperazine-1-carboxylic acid(4-isopropoxyphenyl)-amide (L)-lactate (I) Form 3: Procedure 2

200 mg of 4-[6-methoxy-7-(3-piperidin-1-yl-propoxy)quinazolin-4-yl]piperazine-1-carboxylic acid(4-isopropoxyphenyl)-amide (L)-lactate (I) Form 1 was treated with 2 ml of iPrOAc:THF (1:1)+2.5% water at 0° C. and stirred for 3.5 days. XRPD analysis showed Form 3.

### Example 21

### Preparation of 4-[6-methoxy-7-(3-piperidin-1-yl-propoxy)quinazolin-4-yl]piperazine-1-carboxylic acid(4-isopropoxyphenyl)-amide (L)-lactate (I) Form 4: Procedure 2

4-[6-Methoxy-7-(3-piperidin-1-yl-propoxy)quinazolin-4-yl]piperazine-1-carboxylic acid(4-isopropoxyphenyl)-amide (II) trihydrate (4.0 g, 6.49 mmol) and tetrahydrofuran (17.78 g) were combined and warmed to 40° C. The water content of the solution was measured by Karl-Fischer and water (0.34 ml) was added to bring the water content to 3.2%. An 85% (L)-Lactic acid in water solution (0.741 ml, 8.45 mmol) was added. The solution was seeded with Form 4. After stirring at 40° C. for 1 hour, isopropyl acetate (20 ml) was added over 10 hours, the solution was then held at 40° C. for a further 1 hour, cooled to 30° C. over 2 hours, held 1 hour, cooled to 20° C. over 2 hours and held overnight at this temperature. The suspension was filtered, and the resulting solid dried to constant weight to yield 3.82 g (90% yield) of the title compound.

### Example 22

### Tablet Composition

The composition of the tablet is shown below in Table 7.

TABLE 7
Tablet composition

| Component | Function | mg/Tablet |
|---|---|---|
| Compound of formula (I) | | 308.6 |
| Form 1 | | |
| Calcium Silicate | Disintegrant (intragranular) | 20.0 |
| Crospovidone | Disintegrant (intragranular) | 5.0 |
| Sodium Stearyl Fumarate | Lubricant (intragranular) | 25.0 |
| Microcrystalline Cellulose | Filler (intragranular) | 101.4 |
| Calcium Silicate | Disintegrant (extragranular) | 20.0 |
| Colloidal Silicon Dioxide | Glidant (extragranular) | 5.0 |
| Crospovidone | Disintegrant (extragranular) | 5.0 |
| Sodium Stearyl Fumarate | Lubricant (extragranular) | 10.0 |
| Total tablet weight, mg | | 500.0 |

### Example 23

### Tablet Composition

The composition of the tablet is shown below in Table 8.

TABLE 8
Tablet composition

| | | mg/ |
|---|---|---|
| Ingredients | Function | Tablet |
| Compound of formula (I) Form 1 | | 304.9 |
| Sodium Stearyl Fumarate | Lubricant (intragranular) | 30.0 |
| Crospovidone | Disintegrant (intragranular) | 30.0 |
| Silicified Microcrystalline | Filler (intragranular) | 175.1 |
| Cellulose | | |
| Colloidal Silicon Dioxide | Glidant (extragranular) | 6.0 |
| Crospovidone | Disintegrant (extragranular) | 30.0 |
| Sodium Stearyl Fumarate | Lubricant (extragranular) | 24.0 |
| Total tablet weight, mg | | 600.0 |

### Example 24

### Tablet Composition

The composition of the tablet is shown below in Table 9.

TABLE 9
Tablet composition

| | | mg/ |
|---|---|---|
| Component | Function | Tablet |
| Compound of formula (I) Form 1 | | 299.4 |
| Sodium Stearyl Fumarate | Lubricant (intragranular) | 30.0 |
| Crospovidone | Disintegrant (intragranular) | 30.0 |
| Isomalt | Filler (intragranular) | 66.6 |
| Colloidal Silicon Dioxide | Glidant (extragranular) | 6.0 |
| Crospovidone | Disintegrant (extragranular) | 24.0 |
| Isomalt | Filler (extragranular) | 120.0 |
| Sodium Stearyl Fumarate | Lubricant (extragranular) | 24.0 |
| Total tablet weight, mg | | 600.0 |
| Opadry ® II Yellow Film Coating | Film Coat | 18.0 |

### Example 25

### Tablet Composition

The composition of the tablet is shown below in Table 10.

TABLE 10
Tablet composition

| | | mg/ |
|---|---|---|
| Component | Function | Tablet |
| Compound of formula (I) Form 1 | | 301.2 |
| Sodium Stearyl Fumarate | Lubricant (intragranular) | 30.0 |
| Sodium Starch Glycolate | Disintegrant (intragranular) | 30.0 |
| Mannitol | Filler (intragranular) | 82.8 |
| Colloidal Silicon Dioxide | Glidant (extragranular) | 6.0 |
| Sodium Starch Glycolate | Disintegrant (extragranular) | 24.0 |
| Mannitol | Filler (extragranular) | 120.0 |
| Magnesium Stearate | Lubricant (extragranular) | 6.0 |
| Total tablet weight, mg | | 600.0 |

### Example 26

### Tablet Composition

The composition of the tablet is shown below in Table 11.

TABLE 11
Tablet composition

| | | mg/ |
|---|---|---|
| Component | Function | Tablet |
| Compound of formula (I) Form 1 | | 292.4 |
| Sodium Stearyl Fumarate | Lubricant (intragranular) | 30.0 |
| Crospovidone | Disintegrant (intragranular) | 30.0 |
| Mannitol | Filler (intragranular) | 187.6 |
| Colloidal Silicon Dioxide | Glidant (extragranular) | 6.0 |
| Crospovidone | Disintegrant (extragranular) | 24.0 |
| Sodium Stearyl Fumarate | Lubricant (extragranular) | 30.0 |
| Total tablet weight, mg | | 600.0 |

### Example 27

### Tablet Composition

The composition of the tablet is shown below in Table 12.

TABLE 12
Tablet composition

| | | mg/ |
|---|---|---|
| Component | Function | Tablet |
| Compound of formula (I) Form 1 | | 146.2 |
| Sodium Stearyl Fumarate | Lubricant (intragranular) | 15.0 |
| Crospovidone | Disintegrant (intragranular) | 15.0 |
| Mannitol | Filler (intragranular) | 93.8 |
| Colloidal Silicon Dioxide | Glidant (extragranular) | 3.0 |
| Crospovidone | Disintegrant (extragranular) | 12.0 |
| Sodium Stearyl Fumarate | Lubricant (extragranular) | 15.0 |
| Total tablet weight, mg | | 300.0 |
| Opadry ® II Yellow Film Coating | Film Coat | 9.0 |

### Example 28

### Batch Composition

The compound of formula (I), Form 1, (9.75 kg) was blended with sieved sodium stearyl fumarate (1.0 kg). The resulting mixture was blended with sieved crospovidone (1.00 kg) and sieved mannitol (6.25 kg). The resulting mixture was sieved and further blended, and was then passed through a roller compactor to generate a ribbon which was milled. The resulting granules were blended with sieved crospovidone (0.80 kg) and sieved colloidal silicon dioxide (0.20 kg), and the resulting mixture was blended with sieved sodium stearyl fumarate (1.00 kg) to give a batch with composition as shown in Table 13.

TABLE 13
Batch composition

| | | % (weight/ | kg/ |
|---|---|---|---|
| Component | Function | weight) | batch |
| Compound of formula (I) | | 48.75 | 9.75 |
| Form 1 | | | |
| Sodium Stearyl Fumarate | Lubricant (intragranular) | 5.00 | 1.00 |
| Crospovidone | Disintegrant | 5.00 | 1.00 |
| | (intragranular) | | |
| Mannitol | Filler (intragranular) | 31.25 | 6.25 |
| Crospovidone | Disintegrant | 4.00 | 0.80 |
| | (extragranular) | | |
| Colloidal Silicon Dioxide | Glidant (extragranular) | 1.00 | 0.20 |
| Sodium Stearyl Fumarate | Lubricant (extragranular) | 5.00 | 1.00 |
| Total weight, % or batch | | 100.00 | 20.00 |

6.38 kg of the batch prepared according to Table 13 was taken and compressed into 300 mg tablets. The 300 mg tablets were coated using the Opadry® II Yellow Film Coating System (9 mg/Tablet). 12.76 kg of the batch prepared according to Table 13 was taken and compressed into 600 mg tablets. The 600 mg tablets were coated using the Opadry® II Yellow Film Coating System (18 mg/Tablet).

While we have described a number of embodiments of this invention, it is apparent that our basic examples may be altered to provide other embodiments, which utilize the compounds and methods of this invention. Therefore, it will be appreciated that the scope of this invention is to be defined by the appended claims rather than by the specific embodiments, which have been represented by way of example

What is claimed is:

1. The compound of formula (I): 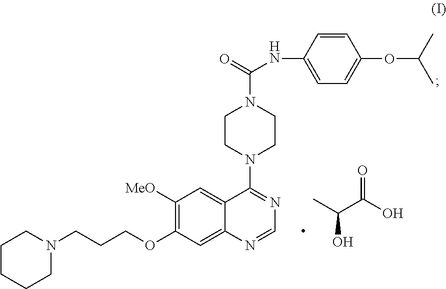
or a crystalline form thereof.

2. The compound of claim 1, wherein at least 95% by weight is crystalline.

3. The compound of claim 1, wherein the crystalline form is Form 1.

4. The compound of claim 3, wherein Form 1 is characterized by at least one X-ray powder diffraction peak at 2θ angles of 5.50°, 10.98°, 19.65°, 19.97°, and 21.83°.

5. The compound of claim 3, wherein Form 1 is characterized by at least one X-ray powder diffraction peak at 2θ angles of 5.50°, 19.65°, and 19.97°.

6. The compound of claim 3, wherein Form 1 is characterized by at least one of the following features (I-i)-(I-iii):
(I-i) at least one of the X-ray powder diffraction peaks shown in Table 1;
(I-ii) an X-ray powder diffraction pattern substantially similar to FIG. 1; and
(I-iii) a differential scanning calorimetry (DSC) profile having an endothermic range of about 175° C. to about 185° C., with an onset temperature of about 177° C.

7. The compound of claim 1, wherein the crystalline form is Form 2.

8. The compound of claim 7, wherein Form 2 is characterized by at least one X-ray powder diffraction peak at 2θ angles of 6.38°, 7.98°, 11.19°, 14.12°, 19.39°, 20.41°, 20.68°, 21.44°, and 27.65°.

9. The compound of claim 7, wherein Form 2 is characterized by at least one X-ray powder diffraction peak at 2θ angles of 11.19°, 19.39°, 20.41°, and 21.44°.

10. The compound of claim 7, wherein Form 2 is characterized by at least one of the following features (II-i)-(II-iii):
(II-i) at least one of the X-ray powder diffraction peaks shown in Table 2;
(II-ii) an X-ray powder diffraction pattern substantially similar to FIG. 4; and
(II-iii) a differential scanning calorimetry (DSC) profile, comprising a first endothermic range of about 150° C. to about 160° C., with an onset temperature of about 155.7° C.

11. The compound of claim 1, wherein the crystalline form is Form 3.

12. The compound of claim 11, wherein Form 3 is characterized by at least one X-ray powder diffraction peak at 2θ angles of 3.66°, 11.04°, 19.93°, and 23.98°.

13. The compound of claim 11, wherein Form 3 is characterized by at least one of the following features, (III-i)-(III-iii):
(III-i) at least one of the X-ray powder diffraction peaks shown in Table 3;
(III-ii) an X-ray powder diffraction pattern substantially similar to FIG. 7; and
(III-iii) a differential scanning calorimetry (DSC) profile substantially similar to FIG. 8.

14. The compound of claim 1, wherein the crystalline form is Form 4.

15. The compound of claim 14, wherein Form 4 is characterized by at least one X-ray powder diffraction peak at 2θ angles of 11.30°, 12.71°, 15.15°, 16.02°, 20.03°, 24.15°, and 24.66°.

16. The compound of claim 14, wherein Form 4 is characterized by at least one X-ray powder diffraction peak at 2θ angles of 11.30°, 16.02°, 20.03°, and 24.15°.

17. The compound of claim 14, wherein Form 4 is characterized by at least one of the following features, (IV-i)-(IV-iii):
(IV-i) at least one of the X-ray powder diffraction peaks shown in Table 4;
(IV-ii) an X-ray powder diffraction pattern substantially similar to FIG. 10; and
(IV-iii) a differential scanning calorimetry (DSC) profile substantially similar to FIG. 11.

18. A pharmaceutical composition comprising the compound of formula (I): 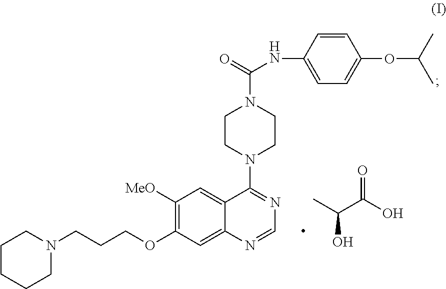
or a crystalline form thereof, a lubricant, a filler, a disintegrant, and a glidant.

19. The pharmaceutical composition of claim 18, wherein the pharmaceutical composition comprises about 30% to about 60% of the compound of formula (I), or a crystalline form thereof; about 6% to about 12% of a lubricant; about 6% to about 12% of a disintegrant; about 15% to about 50% of a filler; and about 0.3% to about 2% of a glidant, by weight as a percentage of total weight.

20. The pharmaceutical composition of claim 18, wherein the pharmaceutical composition comprises about 45% to about 55% of the compound of formula (I), or a crystalline form thereof; about 9% to about 11% of a lubricant; about 8% to about 10% of a disintegrant; about 20% to about 40% of a filler; and about 0.8% to about 1.5% of a glidant, by weight as a percentage of total weight.

21. The pharmaceutical composition of claim 18, wherein the pharmaceutical composition is an oral pharmaceutical dosage form.

22. The pharmaceutical composition of claim 21, wherein the oral pharmaceutical dosage form is a tablet.

23. The pharmaceutical composition of claim 18, wherein the crystalline form is Form 1.

24. The pharmaceutical composition of claim 18, wherein the compound of formula (I), or a crystalline form thereof, is present in an amount of about 30% to about 60%, by weight as a percentage of total weight.

25. The pharmaceutical composition of claim 18, wherein the compound of formula (I), or a crystalline form thereof, is present in an amount of about 44% to about 55%, by weight as a percentage of total weight.

26. The pharmaceutical composition of claim 18, wherein the lubricant is present in an amount of between about 6% to about 12%, by weight as a percentage of total weight.

27. The pharmaceutical composition of claim 18, wherein the lubricant comprises a first lubricant, and a second lubricant, wherein the first and second lubricants are independently magnesium stearate, sodium stearyl fumarate, or mixtures thereof.

28. The pharmaceutical composition of claim 27, wherein the first lubricant is sodium stearyl fumarate, and the second lubricant is sodium stearyl fumarate.

29. The pharmaceutical composition of claim 18, wherein the disintegrant is present in an amount of about 6% to about 12%, by weight as a percentage of total weight.

30. The pharmaceutical composition of claim 18, wherein the disintegrant comprises a first disintegrant, and a second disintegrant, wherein the first and second disintegrants are independently crospovidone, calcium silicate, sodium starch glycolate, or mixtures thereof.

31. The pharmaceutical composition of claim 30, wherein the first disintegrant is crospovidone, and the second disintegrant is crospovidone.

32. The pharmaceutical composition of claim 18, wherein the filler is present in an amount of about 15% to about 50%, by weight as a percentage of total weight.

33. The pharmaceutical composition of claim 18, wherein the filler is microcrystalline cellulose, silicified microcrystalline cellulose, isomalt, mannitol, or mixtures thereof.

34. The pharmaceutical composition of claim 33, wherein the filler is mannitol.

35. The pharmaceutical composition of claim 18, wherein the glidant is present in an amount of about 0.3% to about 2%, by weight as a percentage of total weight.

36. The pharmaceutical composition of claim 18, wherein the glidant is silicon dioxide, colloidal silicon dioxide, talc, tribasic calcium phosphate, starch, magnesium trisilicate, powdered cellulose, or mixtures thereof.

37. The pharmaceutical composition of claim 36, wherein the glidant is colloidal silicon dioxide.

38. The pharmaceutical composition of claim 18, wherein the pharmaceutical composition comprises about 45% to about 55% of the compound of formula (I) Form 1; about 9% to about 11% of sodium stearyl fumarate; about 8% to about 10% of crospovidone; about 20% to about 40% of mannitol; and about 0.8% to about 1.5% of colloidal silicon dioxide, by weight as a percentage of total weight.

39. A process for the bulk production of an oral pharmaceutical dosage form of the compound of formula (I), or a crystalline form thereof, wherein the oral pharmaceutical dosage form is a tablet, comprising the steps of:
(a-1) blending the compound of formula (I), or a crystalline form thereof, and sieved first lubricant;
(a-2) blending the resulting mixture from step (a-1) with sieved first disintegrant, and sieved filler;
(a-3) sieving the resulting mixture from step (a-2), then further blending;
(a-4) roller compacting the resulting mixture from step (a-3) to a ribbon;
(a-5) milling the resulting ribbon from step (a-4);
(a-6) blending the resulting granules from step (a-5) with sieved glidant, and sieved second disintegrant;
(a-7) blending the resulting mixture from step (a-6) with sieved second lubricant;
(a-8) tableting the resulting mixture from step (a-7); and
(a-9) optionally film coating the resulting tablets from step (a-8).

40. The process of claim 39, comprising the steps of:
(b-1) blending the compound of formula (I), or a crystalline form thereof, and sieved sodium stearyl fumarate;
(b-2) blending the resulting mixture from step (b-1), with sieved crospovidone, and sieved mannitol;
(b-3) sieving the resulting mixture from step (b-2), then further blending;
(b-4) roller compacting the resulting mixture from step (b-3) to a ribbon;
(b-5) milling the resulting ribbon from step (b-4);
(b-6) blending the resulting granules from step (b-5) with sieved colloidal silicon dioxide, and sieved crospovidone;
(b-7) blending the resulting mixture from step (b-6) with sieved sodium stearyl fumarate;
(b-8) tableting the resulting mixture from step (b-7); and
(b-9) optionally film coating the resulting tablets from step (b-8).

41. A method for treating cancer, comprising the administration of a therapeutically effective amount of a compound according to claim 1, and a pharmaceutically acceptable carrier or diluent.

42. A method for treating cancer, comprising the administration of a therapeutically effective amount of a pharmaceutical composition according to claim 18.

43. The method of claim 41 or 42, wherein the cancer is AML, or malignant glioma.

44. The method of claim 43, wherein the malignant glioma is glioblastoma multiforme.
